SEQUENCE LISTING (50 sequences)

SEQ ID NO: 1 - protein, 208 aa, Mus musculus
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKS

SEQ ID NO: 2 - protein, 217 aa, Mus musculus
QVQLVQSGAEVRKPGASVKVSCKASGDTFSSYAISWVRQAPGQGLEWMGGIIPIFGTANYAQAFQGRVTITANESTSTAYMELSSLRSEDTAIYYCARDNPTLLGSDYWGAGTLVTVSSASTKGPSVFPLAPCSRSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 3 - protein, 208 aa, Artificial Sequence
  for lipid conjugation
DIQMTQSPSTLSASIGDRVCITCRASEGIYHWLAWYQQKPGKAPKLLIYKASSLASGAPSRFSGSGSGTDFTLTISSLQPDDFATYYCQQYSNYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKS

SEQ ID NO: 4 - protein, 208 aa, Artificial Sequence
  for lipid conjugation
DIQMTQSPSTLSASIGDRVTICCRASEGIYHWLAWYQQKPGKAPKLLIYKASSLASGAPSRFSGSGSGTDFTLTISSLQPDDFATYYCQQYSNYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKS

SEQ ID NO: 5 - protein, 213 aa, Mus musculus
ALQLTQSPSSLSASVGDRITITCRASQGVTSALAWYRQKPGSPPQLLIYDASSLESGVPSRFSGSGSGTEFTLTISTLRPEDFATYYCQQLHFYPHTFGGGTRVDVRRTVAAPSVFIFPPSDEQKSGTASVVCLLNNFYPREAKVQWKVDNALQSGNSQESVTEQDSKDSTYSLSSTLTLSKADYEKHKVYECEVTHQGLSSPVTKSFNRGEC

SEQ ID NO: 6 - protein, 237 aa, Mus musculus
RITLKESGPPLVKPTQTLTLTCSFSGFSLSDFGVGVGWIRQPPGKALEWLAIIYSDDDKRYSPSLNTRLTITKDTSKNQVVLVMTRVSPVDTATYFCAHRRGPTTLFGVPIARGPVNAMDVWGQGITVTISSTSTKGPSVFPLAPSSKSTSGGTAALGCLVKDYFPEPVTVSWNSGALTSGVHTFPAVLQSSGLYSLSSVVTVPSSSLGTQTYTCNVNHKPSNTKVDKRVEPKSCDK

SEQ ID NO: 7 - protein, 213 aa, Artificial Sequence
  for lipid conjugation
ALQLTQSPSSLSASVGDRICITCRASQGVTSALAWYRQKPGSPPQLLIYDASSLESGVPSRFSGSGSGTEFTLTISTLRPEDFATYYCQQLHFYPHTFGGGTRVDVRRTVAAPSVFIFPPSDEQKSGTASVVCLLNNFYPREAKVQWKVDNALQSGNSQESVTEQDSKDSTYSLSSTLTLSKADYEKHKVYECEVTHQGLSSPVTKSFNRGEC

SEQ ID NO: 8 - protein, 213 aa, Artificial Sequence
  for lipid conjugation
ALQLTQSPSSLSASVGDRITICCRASQGVTSALAWYRQKPGSPPQLLIYDASSLESGVPSRFSGSGSGTEFTLTISTLRPEDFATYYCQQLHFYPHTFGGGTRVDVRRTVAAPSVFIFPPSDEQKSGTASVVCLLNNFYPREAKVQWKVDNALQSGNSQESVTEQDSKDSTYSLSSTLTLSKADYEKHKVYECEVTHQGLSSPVTKSFNRGEC

SEQ ID NO: 9 - protein, 237 aa, Artificial Sequence
  non-functional mutant
RITLKESGPPLVKPTQTLTLTCSFSGFSLSDFGVGVGWIRQPPGKALEWLAIIYSDDDKRYSPSLNTRLTITKDTSKNQVVLVMTRVSPVDTATYFCAHRRGPTTSSGVPIARGPVNAMDVWGQGITVTISSTSTKGPSVFPLAPSSKSTSGGTAALGCLVKDYFPEPVTVSWNSGALTSGVHTFPAVLQSSGLYSLSSVVTVPSSSLGTQTYTCNVNHKPSNTKVDKRVEPKSCDK

SEQ ID NO: 10 - protein, 215 aa, Mus musculus
EIVLTQSPGTQSLSPGERATLSCRASQSVGNNKLAWYQQRPGQAPRLLIYGASSRPSGVADRFSGSGSGTDFTLTISRLEPEDFAVYYCQQYGQSLSTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 228 aa, Mus musculus
QVQLVQSGAEVKRPGSSVTVSCKASGGSFSTYALSWVRQAPGRGLEWMGGVIPLLTITNYAPRFQGRITITADRSTSTAYLELNSLRPEDTAVYYCAREGTTGWGWLGKPIGAFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 12 - protein, 215 aa, Artificial Sequence
  for lipid conjugation
EIVLTQSPGTQSLSPGERACLSCRASQSVGNNKLAWYQQRPGQAPRLLIYGASSRPSGVADRFSGSGSGTDFTLTISRLEPEDFAVYYCQQYGQSLSTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 215 aa, Artificial Sequence
  for lipid conjugation
EIVLTQSPGTQSLSPGERATLCCRASQSVGNNKLAWYQQRPGQAPRLLIYGASSRPSGVADRFSGSGSGTDFTLTISRLEPEDFAVYYCQQYGQSLSTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 210 aa, Homo sapiens [sequence omitted]

SEQ ID NO: 15 - protein, 225 aa, Homo sapiens [sequence omitted]

SEQ ID NO: 16 - protein, 210 aa, Artificial Sequence
  for lipid conjugation
EIVLTQSPGTLSLSPGETACISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 17 - protein, 210 aa, Artificial Sequence
  for lipid conjugation
EIVLTQSPGTLSLSPGETAIICCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 18 - protein, 103 aa, Homo sapiens
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIK

SEQ ID NO: 19 - protein, 121 aa, Homo sapiens
QVQLVQSGGQMKKPGESMRISCQASGYEFIDCTLNWVRLAPGRRPEWMGWLKPRGGAVNYARPLQGRVTMTRDVYSDTAFLELRSLTADDTAVYYCTRGKNCDYNWDFEHWGRGTPVTVSS

SEQ ID NO: 20 - protein, 103 aa, Artificial Sequence
  for lipid conjugation
EIVLTQSPGTLSLSPGETACISCRTSQYGSLAWYQQRPGQAPRLVIYSGSTRAAGIPDRFSGSRWGPDYNLTISNLESGDFGVYYCQQYEFFGQGTKVQVDIK

SEQ ID NO: 21 - protein, 103 aa, Artificial Sequence
  for lipid conjugation
EIVLTQSPGTLSLSPGETAIICCRTSQYGSLAWYQQRPGQAPRLVIYSGSTRAAGIPDRFSGSRWGPDYNLTISNLESGDFGVYYCQQYEFFGQGTKVQVDIK

SEQ ID NO: 22 - protein, 221 aa, Mus musculus
QSVLTQPPSVSAAPGQKVTISCSGSSSNIGNDYVSWYQQLPGTAPKLLIYDNNKRPSGIPDRFSGSKSGTSATLGITGLQTGDEANYYCATWDRRPTAYVVFGGGTKLTVLGAAAGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 226 aa, Mus musculus
EVQLVESGAEVKKPGSSVKVSCKASGGPFRSYAISWVRQAPGQGPEWMGGIIPIFGTTKYAPKFQGRVTITADDFAGTVYMELSSLRSEDTAMYYCAKHMGYQVRETMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 24 - protein, 221 aa, Artificial Sequence
  for lipid conjugation
QSVLTQPPSVSAAPGQKVCISCSGSSSNIGNDYVSWYQQLPGTAPKLLIYDNNKRPSGIPDRFSGSKSGTSATLGITGLQTGDEANYYCATWDRRPTAYVVFGGGTKLTVLGAAAGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 221 aa, Artificial Sequence
  for lipid conjugation
QSVLTQPPSVSAAPGQKVTICCSGSSSNIGNDYVSWYQQLPGTAPKLLIYDNNKRPSGIPDRFSGSKSGTSATLGITGLQTGDEANYYCATWDRRPTAYVVFGGGTKLTVLGAAAGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 213 aa, Mus musculus
DIQMTQSPSTLSASVGDRVTITCKCQLSVGYMHWYQQKPGKAPKLLIYDTSKLASGVPSRFSGSGSGTAFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 227 aa, Mus musculus
QVTLRESGPALVKPTQTLTLTCTFSGFSLSTSGMSVGWIRQPPGKALEWLADIWWDDKKDYNPSLKSRLTISKDTSANQVVLKVTNMDPADTATYYCARSMITNWYFDVWGAGTTVTVSSASTKGPSVFPLAPSSAAAAGGTAALGCLVKDYFPEPVTVSWNSGALTSGVHTFPAVLQSSGLYSLSSVVTVPSSSLGTQTYICNVNHKPSNTKVDKRVEPKSCDKTH

SEQ ID NO: 28 - protein, 213 aa, Artificial Sequence
  for lipid conjugation
DIQMTQSPSTLSASVGDRVCITCKCQLSVGYMHWYQQKPGKAPKLLIYDTSKLASGVPSRFSGSGSGTAFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 213 aa, Artificial Sequence
  for lipid conjugation
DIQMTQSPSTLSASVGDRVTICCKCQLSVGYMHWYQQKPGKAPKLLIYDTSKLASGVPSRFSGSGSGTAFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 213 aa, Mus musculus
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 225 aa, Mus musculus
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 32 - protein, 213 aa, Artificial Sequence
  for lipid conjugation
DIQMTQSPSTLSASVGDRVCITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 213 aa, Artificial Sequence
  for lipid conjugation
DIQMTQSPSTLSASVGDRVTICCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 213 aa, Mus musculus
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 224 aa, Mus musculus
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 36 - protein, 213 aa, Artificial Sequence
  for lipid conjugation
QIVLSQSPAILSASPGEKVCMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 213 aa, Artificial Sequence
  for lipid conjugation
QIVLSQSPAILSASPGEKVTMCCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 214 aa, Mus musculus
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 220 aa, Mus musculus
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEP

SEQ ID NO: 40 - protein, 214 aa, Artificial Sequence
  for lipid conjugation
DIQMTQSPSSLSASVGDRVCITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 214 aa, Artificial Sequence
  for lipid conjugation
DIQMTQSPSSLSASVGDRVTICCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 213 aa, Mus musculus
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

SEQ ID NO: 43 - protein, 221 aa, Mus musculus
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKS

SEQ ID NO: 44 - protein, 213 aa, Artificial Sequence
  for lipid conjugation
DILLTQSPVILSVSPGERVCFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

SEQ ID NO: 45 - protein, 213 aa, Artificial Sequence
  for lipid conjugation
DILLTQSPVILSVSPGERVSFCCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

SEQ ID NO: 46 - protein, 22 aa, Mus musculus
  misc_feature (7)..(12): Xaa can be any naturally occurring amino acid
RRGPTTXXXXXXARGPVNAMDV

SEQ ID NO: 47 - protein, 18 aa, Mus musculus
  misc_feature (6)..(11): Xaa can be any naturally occurring amino acid
EGTTGXXXXXXPIGAFAH

SEQ ID NO: 48 - protein, 450 aa, Mus musculus
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDTPPPCPRCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 214 aa, Mus musculus; identical to SEQ ID NO: 38

SEQ ID NO: 50 - protein, 214 aa, Artificial Sequence; identical to SEQ ID NO: 40
  for lipid conjugation

(19) United States
(12) Patent Application Publication
(10) Pub. No.: US 2013/0150563 A1
(43) Pub. Date: Jun. 13, 2013

(54) LIPID-CONJUGATED ANTIBODIES

(71) Applicants: Antonello Pessi, Rome (IT); Alfredo Nicosia, Rome (IT); Riccardo Cortese, Rome (IT)

(72) Inventors: Antonello Pessi, Rome (IT); Alfredo Nicosia, Rome (IT); Riccardo Cortese, Rome (IT)

(73) Assignee: JV BIO SRL, Naplea (IT)

(21) Appl. No.: 13/808,982

(22) PCT Filed: Jul. 8, 2011
(86) PCT No.: PCT/EP2011/003418

§ 371 Date: Feb. 21, 2013

Related U.S. Application Data
(60) Provisional application No. 61/362,820, filed on Jul. 9, 2010.

(51) Int. Cl. C07K 16/08

(52) U.S. Cl. CPC C07K 16/08
USPC 530/391.1

(57) ABSTRACT

The present invention relates to novel lipid-conjugated antibodies for use in the treatment or the prevention of diseases, including but not limited to cancer, metabolic diseases including but not limited to hyperglycemia and diabetes, obesity, hypertension, hypercholesterolemia, allergy, asthma, Alzheimer's disease, and infectious diseases including but not limited to diseases caused by viruses, bacteria and fungi.

The present invention relates to novel lipid-conjugated antibodies for use in the treatment or the prevention of diseases, including but not limited to cancer, metabolic diseases including but not limited to hyperglycemia and diabetes, obesity, hypertension, hypercholesterolemia, allergy, asthma, Alzheimer's disease, and infectious diseases including but not limited to diseases caused by viruses, bacteria and fungi.

### BACKGROUND OF THE INVENTION

Biological membranes play a key role in the physiology and pathology of the cell. A majority of physiological and pathological phenomena are mediated by receptors embedded in, or linked to biological membranes, including both plasma membranes and intracellular membranes. These proteins and protein complexes are often localized to membrane microdomains known as “lipid rafts” which are enriched in particular lipids such as cholesterol and sphyngolipids (B. Alberts et al., Molecular biology of the cell, Fifth Ed., Garland Science 2008).

For example, infection of the cell by “Enveloped viruses”, such as orthomyxoviruses, paramyxoviruses, retroviruses, flaviviruses, rhabdoviruses and alphaviruses, which are surrounded by a lipid bilayer originating from the host plasma membrane (Ono and Freed, Adv. Virus Res., 2005, 273, 5419-5442), requires the viral glycoproteins to bind specific receptors displayed on the plasma membrane and fusion between viral and host cell membranes. Interfering with any of these two steps targeting the viral glycoproteins or the cell-receptors represents a viable strategy for prophylaxis or therapy of viral infections.

In the case of cancer, overexpression of proteins displayed on the plasma membrane is characteristic of tumor cells and may even provide for a selective advantage on tumoral versus normal cells (tumor associated antigens). Also in this case, targeting these membrane-associated proteins represents an effective strategy for anticancer therapy. Licensed vaccines and therapeutic substances for many enveloped viruses are currently not available. Also when available, therapeutic substances against viruses and a variety of other diseases such as cancer, metabolic diseases, obesity, hypertension, hypercholesterolemia, allergy, asthma and Alzheimer's disease still exhibit severe side-effects. This is frequently the case as these pharmaceuticals are applied in substantial doses which are required to interfere with the pathogenic event e.g. viral entry or dysregulation of the cellular machinery.

In summary, there is a need to develop more effective drugs that can thus be administered in smaller doses and that preferably are effective against the aforementioned diseases, offering an effective and universal therapeutic and prophylactic approach.

Improving the binding efficiency of antibodies against viruses or cell surface displayed proteins by building into the antibody the ability to bind the lipid-membrane represents a general approach for generating more effective and better tolerated therapeutic and prophylactic agents.

### BRIEF SUMMARY OF THE INVENTION

Preferred agents that are effective inter alia against cancer, metabolic diseases including but not limited to hyperglycemia and diabetes, obesity, hypertension, hypercholesterolemia, allergy, asthma, Alzheimer's disease, and infectious diseases including but not limited to diseases caused by viruses, bacteria and fungi also include therapeutic as well as prophylactic antibodies.

The present inventors have identified novel and improved antibodies with excellent pharmaceutical properties and considerably improved biological potency. It was shown that antibodies capable of specifically binding their respective epitope could be modified to additionally bind the plasma membrane of a target cell (e.g. a cancer cell) or the plasma membrane of an enveloped pathogenic virus. Surprisingly, such modifications are capable of increasing the potency of the antibodies which allows reducing the dose required to achieve the desired therapeutic effect. Therefore, the objective problem underlying the present invention was solved by covalently linking a lipid, optionally via a linker, to a therapeutic, prophylactic or diagnostic antibody. For the treatment of a disease caused by an enveloped virus it proves to be particularly effective when linking the antibody, optionally via a linker as described herein, to cholesterol or a derivative thereof.

Accordingly, in a first aspect, the invention provides an antibody or fragment thereof, wherein the antibody or the fragment thereof is covalently linked, optionally via a linker, to a lipid, wherein the lipid or said linker is covalently linked to an amino acid of an antibody domain of said antibody or fragment thereof selected from the group consisting of V_{L}, V_{H}, C_{L}, C_{HI}, C_{H2} and C_{H3}.

In a second aspect the invention provides an antibody or fragment according to the invention for use in the treatment or the prevention of a disease selected from the group consisting of cancer, a metabolic disease including but not limited to hyperglycemia and diabetes, obesity, hypertension, hypercholesterolemia, allergy, asthma, Alzheimer's disease and an infectious disease including but not limited to diseases caused by viruses, bacteria and fungi. The antibody or fragment according to the invention can be used in a method of treatment or prevention of a disease selected from the group consisting of cancer, a metabolic disease including but not limited to hyperglycemia and diabetes, obesity, hypertension, hypercholesterolemia, allergy, asthma, Alzheimer's disease and an infectious disease including but not limited to diseases caused by viruses, bacteria and fungi. The antibody or fragment according to the invention can be used in the manufacture of a medicament for the treatment or the prevention of a disease selected from the group consisting of cancer, a metabolic disease including but not limited to hyperglycemia and diabetes, obesity, hypertension, hypercholesterolemia, allergy, asthma, Alzheimer's disease and an infectious disease including but not limited to diseases caused by viruses, bacteria and fungi.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland) and as described in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, edited by Susan Budavari et al., CRC Press, 1996, and the United States Pharmacopeia-25/National Formulary-20, published by the United States Pharmcopeial Convention, Inc., Rockville Md., 2001. The therapeutic and prophylactic antibodies of the invention comprise amino acids which are designated following the standard one- or three-letter code according to WIPO standard ST.25 unless otherwise indicated.

Throughout this specification and the claims which follow, unless the context requires otherwise, the word “comprise”, and variations such as “comprises” and “comprising”, will be understood to imply the inclusion of a stated feature, integer or step or group of features, integers or steps but not the exclusion of any other feature, integer or step or group of integers or steps. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The term “antibody or fragment thereof”, as used herein, refers to immunoglobulin molecules and immunologically active portions of immunoglobulin molecules, i.e. molecules that contain an antigen binding site that specifically binds an antigen. Also comprised are immunoglobulin-like proteins that are selected through techniques including, for example, phage display to specifically bind to a target molecule or target protein. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. “Antibodies and fragments thereof” suitable for use in the present invention include, but are not limited to, polyclonal, monoclonal, monovalent, bispecific, heteroconjugate, multispecific, human, humanized (in particular CDR-grafted), deimmunized, or chimeric antibodies, single chain antibodies (e.g. scFv), Fab fragments, F(ab′)₂ fragments, fragments produced by a Fab expression library, diabodies or tetrabodies (Holliger P. et al., 1993), nanobodies, anti-idiotypic (anti-Id) antibodies (including, e.g., anti-Id antibodies to antibodies of the invention), and epitope-binding fragments of any of the above.

In some embodiments the antibody fragments are mammalian, preferably human antigen-binding antibody fragments of the present invention and include, but are not limited to, Fab, Fab′ and F(ab′)₂, Fd, single-chain Fvs (scFv), single-chain antibodies, disulfide-linked Fvs (dsFv) and fragments comprising either a VL or VH domain. Antigen-binding antibody fragments, including single-chain antibodies, may comprise the variable domain(s) alone or in combination with the entirety or a portion of the following: hinge region, CL, CH1, CH2, and CH3 domains. Also included in the invention are antigen-binding fragments also comprising any combination of variable domain(s) with a hinge region, CL, CH1, CH2, and CH3 domains.

Antibodies usable in the invention may be from any animal origin including birds and mammals. Preferably, the antibodies are human, simian (e.g. chimpanzee, bonobo, macaque), rodent (e.g. mouse and rat), donkey, sheep rabbit, goat, guinea pig, camel, horse, or chicken. It is particularly preferred that the antibodies are of human or murine origin. As used herein, “human antibodies” include antibodies having the amino acid sequence of a human immunoglobulin and include antibodies isolated from human immunoglobulin libraries or from animals transgenic for one or more human immunoglobulin and that do not express endogenous immunoglobulins, as described for example in U.S. Pat. No. 5,939,598 by Kucherlapati & Jakobovits.

In the context of this invention the unique part of an antigen recognized by an antibody or fragment thereof of the invention is called an “epitope”. The different regions that an antibody comprises are well known in the art and are described e.g. in Janeway C A, Jr et al. (2001), Immunobiology, 5th ed., Garland Publishing.

As used herein, an antibody or antibody fragment of the invention is considered to “specifically bind” to a second compound (e.g. an antigen, such as a target protein), if it has a dissociation constant K_{D} to said second compound of 100 μM or less, preferably 50 μM or less, preferably 30 μM or less, preferably 20 μM or less, preferably 10 μM or less, preferably 5 μM or less, more preferably 1 μM or less, more preferably 900 nM or less, more preferably 800 nM or less, more preferably 700 nM or less, more preferably 600 nM or less, more preferably 500 nM or less, more preferably 400 nM or less, more preferably 300 nM or less, more preferably 200 nM or less, even more preferably 100 nM or less, even more preferably 90 nM or less, even more preferably 80 nM or less, even more preferably 70 nM or less, even more preferably 60 nM or less, even more preferably 50 nM or less, even more preferably 40 nM or less, even more preferably 30 nM or less, even more preferably 20 nM or less, and even more preferably 10 nM or less.

“Pharmaceutically acceptable” means approved by a regulatory agency of the Federal or a state government or listed in the U.S. Pharmacopeia or other generally recognized pharmacopeia for use in animals, and more particularly in humans.

As used herein, the term “protein”, “peptide”, “polypeptide”, “peptides” and “polypeptides” are used interchangeably throughout. These terms refer to both naturally occurring peptides and synthesized peptides that may include naturally or non-naturally occurring amino acids. Peptides can be also chemically modified by modifying a side chain or a free amino or carboxy-terminus of a natural or non-naturally occurring amino acid. This chemical modification includes the addition of further chemical moieties as well as the modification of functional groups in side chains of the amino acids, such as a glycosylation. A peptide is a polymer preferably having at least 3, 4, 5, 6, 7, 8, 9, 10, 15, 20, 25, 30, 35, 40, 45, 50, 55, 60, 65, 70, 75, 80, 85, 90, 95, or at least 100 amino acids, most preferably at least 30 amino acids.

As used herein, the term “carbohydrate” refers to any organic compound including but not limited to monosaccharides, disaccharides, oligosaccharides, and polysaccharides, preferably N-linked and O-linked oligosaccharides and polysaccharides as are well known in the field of antibody research which may comprise hexose-molecules, deoxyhexose molecules, aminohexose molecules, aminononulosonic acid, sialic acid, pentose molecules such as xylose and other molecules including those which are typically comprised in glycosylated proteins.

The term “CDR” in the context of the antibody of the invention or fragment thereof, refers to any of the antibodies complementarity determining regions. In the variable (V) domain of an antibody there are three CDRs (CDR1, CDR2 and CDR3). Since antibodies are typically composed of two polypeptide chains, there is a frequency of about six CDRs for each antigen receptor that can come into contact with the antigen (each heavy and light chain contains three CDRs). Among these, CDR3 shows the greatest variability. CDR domains have been extensively studied and, thus, the average skilled person is well capable of identifying CDR regions, i.e. CDR1, CDR2 and CDR3 within a polypeptide sequence of a VL and VH domain of an antigen receptor. In one preferred method, the CDR1, CDR2 and CDR3 regions of the VL domain are determined as follows:

CDR1 of the VL Domain:

The first amino acid of CDR1 is located at approx. residue 23 or 24 of the VL domain. The residue before the first amino acid of the CDR1 is a conserved Cys residue. The residues following the last amino acid of the CDR1 region is a conserved Trp residue followed typically by Tyr-Gln, but also, Leu-Gln, Phe-Gln or Tyr-Leu. The length of the CDR1 of the VL domain is between 10 and 17 residues.

CDR2 of the VL Domain:

CDR2 is generally located 16 residues after the end of CDR1. The residues before the first amino acid of CDR2 are generally Ile-Tyr, but also, Val-Tyr, Ile-Lys, Ile-Phe or similar. The length of the CDR2 region is generally 7 residues.

CDR3 of the VL Domain:

CDR3 region of the VL domain starts 33 residues after the end of the CDR2 region. The preceeding residue before the first amino acid of CDR3 is always Cys. CDR3 is followed by the amino acids Phe-Gly-XXX-Gly. The length of the CDR3 region is typically between 7 to 11 residues.

In one preferred method, the CDR1, CDR2 and CDR3 regions of the VH domain are determined as follows:

CDR1 of the VH Domain:

The first amino acid of CDR1 is located at approx. residue 26 of the VH domain (always 4 or 5 residues after a Cys). The amino acid after the CDR1 will be a Trp (Typically Trp-Val, but also, Trp-Ile or Trp-Ala). The length of the CDR1 of the VH domain is between 10 to 12 residues.

CDR2 of the VH Domain:

The CDR2 domain starts at residue 15 after the end of the CDR1 of the VH domain. The CDR2 domain is preceeded typically by the amino acids Leu-Glu-Trp-Ile-Gly or a variation thereof. The CDR2 domain will be followed by the three amino acids (Lys/Arg)-(Leu/IleNal/Phe/Thr/Ala)-(Thr/Ser/Ile/Ala) and comprises a total of about 16 to 19 residues.

CDR3 of the VH Domain:

The first amino acid of the CDR3 of the VH domain will be located 33 residues after the end of the CDR2 of the VH domain and will start always 3 amino acids after a conserved Cys residue (the preceding sequence is typically Cys-Ala-Arg). The residues following the CDR3 will be Trp-Gly-XXX-Gly. The CDR3 of the VH domain will typically have a length of between 3 to 25 residues.

The following Table 1 provides an overview over the antibodies referred to herein:

TABLE 1

| Antibody | Target specifically bound by the Antibody |
|---|---|
| MAB CR6261 | Hemagglutinin of influenza A virus |
| MAB D5 | gp41 of HIV |
| MAB 2F5 | gp41 of HIV |
| MAB 4E10 | gp41 of HIV |
| MAB VRC01 | gp120 of HIV |
| MAB VRC02 | gp120 of HIV |
| MAB PALIVIZUMAB | Protein F of respiratory syncytial virus |
| MAB MOTAVIZUMAB | Protein F of respiratory syncytial virus |
| MAB RITUXIMAB | CD20 |
| MAB TRASTUZUMAB | HER2/ErbB2 |
| Mab CETUXIMAB | Epidermal Growth Factor Receptor (EGFR) |

The following Table 2 provides an overview over the sequences referred to herein:

TABLE 2

| SEQ | |
|---|---|
| ID NO: | Description |
| 1 | FAB D5 LIGHT CHAIN |
| 2 | FAB D5 HEAVY CHAIN |
| 3 | LIGHT CHAIN MUTANT A (THR20CYS) OF FAB |
| | D5 FOR LIPID CONJUGATION |
| 4 | LIGHT CHAIN MUTANT B (THR22CYS) OF FAB |
| | D5 FOR LIPID CONJUGATION |
| 5 | FAB 2F5 LIGHT CHAIN |
| 6 | FAB 2F5 HEAVY CHAIN |
| 7 | LIGHT CHAIN MUTANT A (THR20CYS) OF FAB |
| | 2F5 FOR LIPID CONJUGATION |
| 8 | LIGHT CHAIN MUTANT B (THR22CYS) OF FAB |
| | 2F5 FOR LIPID CONJUGATION |
| 9 | HEAVY CHAIN CDR3 DOUBLE MUTANT OF FAB 2F5 |
| 10 | FAB 4E10 LIGHT CHAIN |
| 11 | FAB 4E10 HEAVY CHAIN |
| 12 | LIGHT CHAIN MUTANT A (THR20CYS) OF FAB |
| | 4E10 FOR LIPID CONJUGATION |
| 13 | LIGHT CHAIN MUTANT B (SER22CYS) OF FAB |
| | 4E10 FOR LIPID CONJUGATION |
| 14 | FAB VRC01 LIGHT CHAIN |
| 15 | FAB VRC01 HEAVY CHAIN |
| 16 | LIGHT CHAIN MUTANT A (ILE20CYS) OF FAB |
| | VRC01 FOR LIPID CONJUGATION |
| 17 | LIGHT CHAIN MUTANT B (SER22CYS) OF FAB |
| | VRC01 FOR LIPID CONJUGATION |
| 18 | FAB VRC02 VL |
| 19 | FAB VRC02 VH |
| 20 | VL MUTANT A (ILE20CYS) OF FAB VRC02 |
| | FOR LIPID CONJUGATION |
| 21 | VL MUTANT B (SER22CYS) OF FAB VRC02 FOR |
| | LIPID CONJUGATION |
| 22 | FAB CR6261 LIGHT CHAIN |
| 23 | FAB CR6261 HEAVY CHAIN |
| 24 | LIGHT CHAIN MUTANT A (THR19CYS) OF FAB |
| | CR6261 FOR LIPID CONJUGATION |
| 25 | LIGHT CHAIN MUTANT B (SER21CYS) OF FAB |
| | CR6261 FOR LIPID CONJUGATION |
| 26 | FAB PALIVIZUMAB LIGHT CHAIN |
| 27 | FAB PALIVIZUMAB HEAVY CHAIN |
| 28 | LIGHT CHAIN MUTANT A (THR20CYS) OF FAB |
| | PALIVIZUMAB FOR LIPIDCONJUGATION |
| 29 | LIGHT CHAIN MUTANT B (THR22CYS) OF FAB |
| | PALIVIZUMAB FOR LIPIDCONJUGATION |
| 30 | FAB MOTAVIZUMAB LIGHT CHAIN |
| 31 | FAB MOTAVIZUMAB HEAVY CHAIN |
| 32 | LIGHT CHAIN MUTANT A (THR20CYS) OF FAB |
| | MOTAVIZUMAB FOR LIPIDCONJUGATION |
| 33 | LIGHT CHAIN MUTANT B (THR22CYS) OF FAB |
| | MOTAVIZUMAB FOR LIPIDCONJUGATION |
| 34 | FAB RITUXIMAB LIGHT CHAIN |
| 35 | FAB RITUXIMAB HEAVY CHAIN |
| 36 | LIGHT CHAIN MUTANT A (THR20CYS) OF FAB |
| | RITUXIMAB FOR LIPIDCONJUGATION |
| 37 | LIGHT CHAIN MUTANT B (THR22CYS) OF FAB |
| | RITUXIMAB FOR LIPIDCONJUGATION |
| 38 | FAB TRASTUZUMAB LIGHT CHAIN |
| 39 | FAB TRASTUZUMAB HEAVY CHAIN |
| 40 | LIGHT CHAIN MUTANT A (THR20CYS) OF FAB |
| | TRASTUZUMAB FOR LIPIDCONJUGATION |
| 41 | LIGHT CHAIN MUTANT B (THR22CYS) OF FAB |
| | TRASTUZUMAB FOR LIPIDCONJUGATION |
| 42 | FAB CENTUXIMAB LIGHT CHAIN |
| 43 | FAB CENTUXIMAB HEAVY CHAIN |
| 44 | LIGHT CHAIN MUTANT A (SER20CYS) OF FAB |
| | CENTUXIMAB FOR LIPIDCONJUGATION |
| 45 | LIGHT CHAIN MUTANT B (SER22CYS) OF FAB |
| | CENTUXIMAB FOR LIPIDCONJUGATION |
| 46 | MAB 2F5 HEAVY CHAIN CDR3 |
| 47 | MAB 4E10 HEAVY CHAIN CDR3 |
| 48 | MAB TRASTUZUMAB HEAVY CHAIN |
| 49 | MAB TRASTUZUMAB LIGHT CHAIN |
| 50 | LIGHT CHAIN MUTANT A (THR20CYS) OF MAB |
| | TRASTUZUMAB FOR LIPIDCONJUGATION |

The present inventors have identified novel lipid-conjugated antibodies with improved potency. For example, antibodies that function as inhibitors of viral fusion could be rendered more effective when linked to a lipid. Without being bound by theory it is assumed that antibodies that are modified by linking them to a lipid exhibit an improved partition ratio between antibody in the extracellular medium and antibody bound to a lipid membrane such as the membrane of a cell or an enveloped virus particle, for example. As an example, the antibodies and fragments thereof of the invention preferably localize to the plasma membrane especially to lipid-raft microdomains of the plasma membrane, where they can e.g. block viral entry much more effectively. This permits the application of reduced amounts of therapeutic and prophylactic antibodies to achieve the same health benefit at that low dose that is achieved by a respective non-modified antibody of the state of the art at a respectively larger dose. Furthermore, the lipid moiety of the antibodies of the invention could also aid the cellular uptake of these antibodies, e.g. allowing transporting a therapeutic cargo or even a cytotoxic cargo (suitable to specifically remove e.g. cancer cells) into a target cell.

In a first aspect, the invention provides an antibody or fragment thereof, wherein the antibody or the fragment thereof is covalently linked, optionally via a linker, to a lipid, wherein the lipid or said linker is covalently linked to an amino acid of an antibody domain of said antibody or fragment thereof selected from the group consisting of V_{L}, V_{H}, C_{L}, C_{H1}, C_{H2} and C_{H3}.

Antibodies of the invention and fragments thereof can in preferred embodiments be modified to enhance stability and to enhance antigen binding. Factors affecting stability include exposure of hydrophobic residues that are hidden at the interface of a whole Ig molecule at the constant domain interface; hydrophobic region exposure on the Fv surface leading to intermolecular interaction; and hydrophilic residues in the interior of the Fv beta sheet or at the normal interface between VH and VL (Chowdhury et al., Engineering scFvs for Improved Stability, p. 237-254 in Recombinant Antibodies for Cancer Therapy Methods and Protocols, (Eds. Welschof and Krauss) Humana Press, Totowa, N.J., 2003.). Stability can be enhanced by substituting problematic residues impacting on stability. Such modifications can be achieved by e.g. effecting up to one, two, three, four, five, six, seven, eight, nine or up to ten single amino acid substitutions, deletions, modifications and/or insertions, preferably up to three and most preferably a single substitution, deletion, modification and/or insertion in a polypeptide chain of the antibody or fragment thereof of the invention. Techniques for enhancing single chain antibody stability taking into account problematic residues are well known in art. (Chowdhury et al., Engineering scFvs for Improved Stability, p. 237-254 in Recombinant Antibodies for Cancer Therapy Methods and Protocols, (Eds. Welschof and Krauss) Humana Press, Totowa, N.J., 2003.)

In a preferred embodiment, the antibody of the invention is selected from the group consisting of a polyclonal antibody, a monoclonal antibody, a chimeric antibody, a humanized antibody, a human antibody, a diabody, a tetrabody, a nanobody, a chimeric antibody, and a deimmunized antibody.

In a preferred embodiment, the fragment of the antibody of the invention is an antibody fragment selected from the group consisting of Fab, F(ab′)₂, Fd, Fv, single-chain Fv, and disulfide-linked Fvs (dsFv). An antibody of the invention or a fragment thereof is preferably capable of binding to a lipidmembrane. Furthermore, the antibody or fragment of the invention is preferably capable of:

(i) being internalized into a cell;

(ii) binding to the plasma membrane of a cell and/or

(iii) binding to the lipid membrane of an enveloped virus.

The lipid of the antibody or fragment thereof of the invention will in preferred embodiments as mentioned above allow it bind to a plasma membrane via lipid rafts, and/or to be internalized into a cell preferably via lipid rafts. Many pathogens enter cells via lipid rafts such as the influenza virus so that it is advantageous if an antibody of the invention exhibits the ability of neutralizing such pathogens not only on the cell surface but also intracellularly. Internalization can be studied be several approaches such as those described in Dyer & Benjamins, J. Neurosci. (1988) 883-891, D. C. Blakeyl et al., J. Cell Biochem. Biophys. 24-25 (1994) 175-183, Coffey et al., J. Pharmacol. Exp. Ther. 310 (2004) 896-904.

The average skilled person is also well capable of testing, without undue burden, if an antibody or fragment thereof binds to a lipid membrane e.g. of a cell or of an enveloped virus such as e.g. HIV, Influenza virus, Hepatitis B virus, Hepatitis C virus, Rhinovirus, Herpes virus, Herpes simplex virus, West Nile Virus, Dengue virus, SARS-CoV, Varicella-zoster virus, Pseudorabies virus, Vesicular stomatits virus, Borna disease virus, Newcastle disease virus, Vaccinia virus, Rotavirus, Sendai virus, Measles virus, Mumps virus, Human Parainfluenza virus, Respiratory syncytical virus, Hendra virus, Nipah virus, Ebola virus, Marburg virus, Junin virus, Machupo virus, Guanairito virus or Lassa virus.

For such analysis various tools such as fluorescence-based methods (e.g. colocalization studies, quenching e.t.c), electron microscopy studies and the like are readily available and suitable.

In one embodiment of the antibody or fragment thereof of the invention, the antibody binds, preferably specifically binds, in addition to the lipid membrane (e.g. plasma membrane), also to a polypeptide selected from the group consisting of HIV gp41 (e.g. accession number AAA19156.1), HIV gp120, influenza hemagglutinin (e.g. accession number AAA43099.1 or CAA40728.1), protein F of paramyxoviruses (e.g. accession number AAV54052.1), protein GP2 of filoviruses (e.g. accession number Q89853.1 or AAV48577.1), protein E of flaviviruses (e.g. accession number AAR87742.1), protein S of coronaviruses (e.g. accession number AAP33697.1 or BAC81404.1) and protein G2 of arenaviruses (e.g. accession number BAA00964.2 or P03540.

In another embodiment of the antibody or fragment thereof of the invention, the antibody binds, preferably specifically binds, in addition to the lipid membrane (e.g. plasma membrane), also to a polypeptide selected from the group consisting of HER2, epidermal growth factor receptor (EGFR), CD20, vascular endothelial growth factor (VEGF), tumor necrosis factor (TNF), and scavanger receptor B1 (SR-B 1).

It is within the skill of the artisan to experimentally determine also by other means, if an antibody or fragment thereof of the invention binds to an antigen such as one of the aforementioned polypeptides. For example, it is possible to analyze the interaction between the antibody or fragment thereof and the polypeptide or protein using a pull down assay. For example, the polypeptide may be purified and immobilized on a solid phase such as beads. In one embodiment, the beads linked to the polypeptide may be contacted with the antibody or fragment thereof, washed and probed with a secondary antibody specific for an invariant part of the antibody or fragment thereof, available in the state of the art. Also other binding assays well known in the art and suitable to determine binding affinities between two binding partners can be used such as e.g. ELISA-based assays, fluorescence resonance energy transfer (FRET)-based assays, co-immunoprecipitation assays and plasmon-resonance assays. The binding can be detected by fluorescence means, e.g. using a fluorescently labelled secondary antibody, or enzymatically as is well known in the art. Also radioactive assays may be used to assess binding. Thus, any of the aforementioned exemplary methods can be used to determine if an antibody or fragment thereof of the invention binds to a specific polypeptide and optionally also to determine with what dissociation constant K_{D} the antibody or fragment thereof binds the mentioned antigen.

The lipid of the antibody or fragment thereof of the invention is linked (optionally via a linker) to an amino acid of said antibody or fragment thereof. Preferably, the amino acid is located:
(1) N-terminal to the CDR-1 region of the VL domain of said antibody or fragment thereof;
(2) N-terminal to the CDR-1 region of the VH domain of said antibody or fragment thereof;
(3) within the CDR-3 region of the VL domain of said antibody or fragment thereof; or
(4) within the CDR-3 region of the VH domain of said antibody or fragment thereof.

As described above the CDR-regions of antibodies are well characterized in the art and can be determined by the skilled person for any antibody or antibody-fragment. Examples 9-11 and FIGS. 8-16 as shown below specify particularly preferred amino acids of the light and heavy chain of the Fab-fragment of an antibody that can be used to covalently attach the lipid (optionally via a linker).

Preferred locations of the amino acid are:
(1) at position 20 or 22 of the VL domain of said antibody or fragment thereof;
(2) at position 19 or 21 of the VL domain of said antibody or fragment thereof;
(3) at position 7 or 25 of the VH domain of said antibody or fragment thereof;
(4) at position 197 of the CL domain of said antibody or fragment thereof;
(5) at position 125 of the CH1 domain of said antibody or fragment thereof;
(6) at position 248 or 326 of the CH2 domain of said antibody or fragment thereof; or
(7) at position 415 or 442 of the CH3 domain of said antibody or fragment thereof.
   Most preferred positions are position 19, 20, 21 and 22 of the VL domain. As used herein “position” refers to the location of said amino acid within the heavy or light chain of the antibody or fragment thereof. The position specifies an amino acid which is located at the indicated number of amino acids downstream of the first N-terminal amino acid of the respective light or heavy chain of said antibody or fragment thereof. As mentioned several examples of preferred Fab-fragements and their respective preferred locations of the amino acid are provided in examples 9-11 below (see also FIGS. 8-16). Using sequence alignments the average skilled artisan is well capable of determining these and the aforementioned amino acid positions within the VL or VH domain of any given antibody, preferably counted from the N-terminus of said VL or VH domain.

As used herein “lipid” can be any lipid as long as it has the capability to insert into a cell membrane or an equivalent lipid bilayer. Preferably, a “lipid” and derivatives thereof are capable of integrating into and/or forming rafts as described in Xu, J. Biol. Chem. 276, (2001) 33540-33546 and Wang, Biochemistry 43, (2004) 1010-8.

Preferred embodiments of the invention include an antibody or fragment thereof of the invention, wherein the lipid is selected from the group consisting of cholesterol, a sphingolipid, a glycolipid, a glycerophospholipid and a derivative or pharmaceutically acceptable salt thereof.

In a preferred embodiment of the antibody or fragment thereof; the lipid is a glycolipid selected from the group consisting of a ganglioside, a cerebroside, a globoside and a sulfatide. The ganglioside may be e.g. selected from the group consisting of GD1a, GD1b, GM1, GD3, GM2, GM3, GQ1a and GQ1b.

In another preferred embodiment of the antibody or fragment thereof, the lipid is sphingomyelin or ceramide. If the lipid is a glycerophospholipid then in a preferred embodiment it is selected from the group of glycerophospholipids consisting of phosphatidylcholine, phosphatidylethanolamine and phosphatidylserine.

Cholesterol is capable of inserting into a cell membrane. This property appears to be at least in part responsible for the advantageous properties of the antibodies and fragments thereof according to the invention. Accordingly, also an antibody or fragment thereof of the invention is preferred wherein the lipid is cholesterol or a derivative of cholesterol. Such derivatives are structurally related to cholesterol in that they have the same steroid basic structure, i.e. (8R,9S,10R,13S,14S)-10,13-dimethyl-1,2,6,7,8,9,11,12,14,15,16,17-dodecahydrocyclopenta[a]phenanthren and preferably have a comparable ability to insert into a lipid bilayer having a lipid composition as found in human cells. Preferred integrating derivatives of cholesterol include ergosterol, 7-dihydrocholosterol and stigmasterol. The ability to insert into a lipid bilayer can be tested by art known methods using, e.g. fluorescently labelled cholesterol and structural derivatives thereof on an artificial lipid bilayer. In a preferred embodiment, an integrating derivative of a lipid useful in the invention has the ability to integrate into a lipid raft comprised in a cell membrane. A lipid that can integrate into a lipid raft can generally also form one. Thus, if a lipid can integrate into and thus form a lipid raft can be tested e.g. as described in Xu, J. Biol. Chem. 276, (2001) 33540-33546, Wang, Biochemistry 43, (2004) 1010-8.

Preferably, the lipid that is covalently linked to the antibody or fragment thereof, optionally via a linker, is not polar, e.g. it does not comprise any charged substituents. Furthermore it is preferred that as also mentioned above said lipid is capable of segregating into a lipid raft domain of a cell membrane. That means that the lipid useful for the invention preferably selectively accumulates in lipid rafts. Whether a lipid segregates into lipid rafts can easily be tested as mentioned above or e.g. by contacting a cell with a lipid comprising a radioactive isotope and then isolating lipid-raft microdomains from these cells e.g. by sucrose-gradient centrifugation as described in RADEVA et al., Biochem. J. (2004) 380, p. 219-230 and in Kim et al., “The Isolation of Detergent-Resistant Lipid Rafts for Two-Dimensional Electrophoresis”, Methods in Molecular Biology (2008), Volume 424, p. 413-422. Preferred lipids of the invention that specifically bind to and/or segregate into lipid-raft microdomains will thus co-fractionate with lipid-rafts. In the aforementioned example the radioactive lipid will be detected in the isolated lipid-rafts, i.e. the total amount of radioactivity present in the lipid-raft fraction will be greater than the remaining lipid fraction not isolated with the rafts.

In a preferred embodiment the lipid is a fluorescent lipid, preferably a fluorescent lipid capable of inserting into lipid rafts, and most preferably a lipid having a structure according to any of formulas (I)-(III):

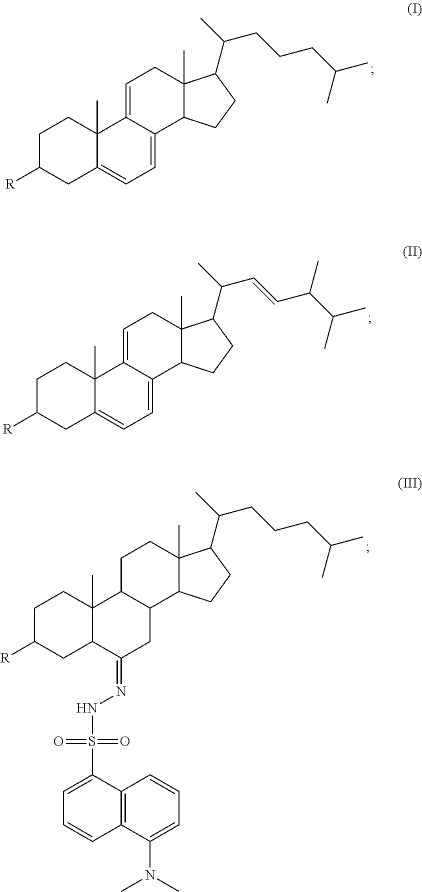

wherein R designates the bond to said linker (if present) or to an amino acid of the antibody or fragment thereof and preferably to a sulphur moiety (e.g. sulfhydryl group) of said amino acid.

The term “covalently linked” refers to a covalent bond between an amino acid of the antibody or fragment thereof and the lipid, e.g. cholesterol or said linker as described herein that may be placed between the antibody or fragment thereof and said lipid.

In preferred embodiments of the antibody or fragment thereof said lipid is selected from a sphingolipid, a glycolipid and a glycerophospholipid that is covalently linked via a free —OH, —NH₃ or —COOH group of the lipid, optionally via said linker, to the C-terminus of the light or heavy chain of said antibody or fragment thereof of the invention.

Preferably, the lipid is a sphingolipid having a structure according to formula IV:

| | |
|---|---|
| | (IV) |
| 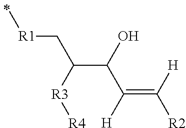 | |

wherein
*denotes where the lipid is attached to the linker or to said amino acid of
said antibody or fragment thereof of the invention and wherein R1 through
R4 are selected from the following list:

| R1 | R2 | R3 | R4 |
|---|---|---|---|
| COCH₂CH₂COO | (CH₂)₁₂CH₃ | NHCO | (CH₂)₁₄CH₃ |
| COCH₂O | (CH₂)₁₂CH₃ | NHCO | (CH₂)₁₄CH₃ |
| COCH₂CH₂COO | (CH₂)₁₂CH₃ | NHCO | (CH₂)₁₄CH₃ |
| COCH₂CH₂COO | (CH₂)₁₂CH₃ | NHCO | (CH₂)₁₈CH₃ |
| COCH₂CH₂COO | (CH₂)₁₂CH₃ | NHCO | (CH₂)₇CHCH(CH₂)₅CH₃ |
| COCH₂CH₂COO | (CH₂)₁₇CH3 | NHCO | (CH₂)₂₈CH₃ |
| COCH₂CH₂CONH | (CH₂)₁₂CH₃ | NHCO | (CH₂)₁₄CH₃ |
| COCH₂CH₂COO | (CH₂)₁₂CH₃ | NH | (CH₂)₁₅CH₃ |
| COCH₂CH₂COO | (CH₂)₁₂CH₃ | NHSO₂ | (CH₂)₁₄CH₃ |
| COCH₂CH₂COO | (CH₂)₁₂CH₃ | NHCONH | (CH₂)₁₇CH₃ |
| COCH₂CH₂COO | (CH₂)₁₇CH₃ | OCO | (CH₂)₂₈CH₃ |
| COCH₂CH₂COO | (CH₂)₁₂CH₃ | NHCONH | (CH₂)₁₅CH₃ |

The antibody or fragment thereof of the invention also includes pharmaceutically acceptable salts thereof. The term “pharmaceutically acceptable salt” refers to a salt of a compound as specified in this patent application including acid addition salts which may, for example, be formed by mixing a solution of the antibody or fragment thereof of the present invention or its lipid with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the antibody or fragment thereof of the invention carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, Berge, S. M., et al, “Pharmaceutical Salts”, Journal of Pharmaceutical Science, 1977, 66, 1-19). Antibodies of the invention and fragments thereof generally contain both basic and acidic functionalities, e.g. Glu, Asp, Gln, Asn, Lys, or Arg, that allow the compounds to be converted into either base or acid addition salts.

The term linker preferably refers to an organic molecule that adopts a linear conformation. Said linker, which is preferably a non-cleavable linker, has a preferred length of between 0.4 nm and 15 nm. This specific preferred range of the length of the linker confers optimal activity (e.g. antiviral activity) to an antibody and fragment of the invention. Thus, it is preferred in this context that the linker has length of between 0.4 nm and 15 nm and that the antibody specifically binds to a polypeptide selected from the group consisting of HIV gp41 (e.g. accession number AAA19156.1), HIV gp120, influenza hemagglutinin (e.g. accession number AAA43099.1 or CAA40728.1), protein F of paramyxoviruses (e.g. accession number AAV54052.1), protein GP2 of filoviruses (e.g. accession number Q89853.1 or AAV48577.1), protein E of flaviviruses (e.g. accession number AAR87742.1), protein S of coronaviruses (e.g. accession number AAP33697.1 or BAC81404.1) and protein G2 of arenaviruses (e.g. accession number BAA00964.2 or P03540.

It is also preferred in this contex that the linker has length between 0.4 nm and 15 nm and that, the antibody specifically binds to a polypeptide selected from the group consisting of HER2, epidermal growth factor receptor (EGFR), CD20, vascular endothelial growth factor (VEGF), tumor necrosis factor (TNF), and scavanger receptor B1 (SR-B1).

Typical linkers of preferred embodiment of the antibody or fragment thereof of the invention may contain a polymeric spacer unit, preferably having between 1 to 30 repeats of a given monomer, and at one end of the spacer unit a moiety that allows linkage to an amino acid, preferably an amino acid containing a chemical functionality like —SH, —OH, —COOH, —NH₂, —HC═O, —RC═O, —O—NH₂, —N═N═N, —C═C—, —C≡C, or —NH—NH₂ and at the other end a moiety allowing linkage to said lipid, e.g. cholesterol or a derivative thereof, preferably via the 3-oxygen moiety of the steroid structure.

Preferably, the lipid or linker of the antibody or fragment thereof according to the invention is covalently linked to said antibody or fragment thereof via a bond selected from the group consisting of an amide bond, an ester bond, a thioether bond, a thioester bond, an aldehyde bond and an oxyme bond. Examples of non-cleavable linker systems which can be used in this invention include the carbodiimide (EDC), the sulfhydryl-maleimide, and the periodate systems, which are all well known in the art. In the carbodiimide system, a water soluble carbodiimide reacts with carboxylic acid groups of the lipid or antibody or fragment thereof, resulting in the activation of this carboxyl group. The carboxyl group is subsequently coupled to an amino group present on the lipid or antibody or fragment thereof. The result of this reaction is a noncleavable amide bond between the lipid and the antibody or fragment thereof. In the sulfhydryl-maleimide system, a sulfhydryl group is for example introduced onto an amine group of the antibody or fragment thereof using a compound such as Traut's reagent. The lipid or linker including the lipid is then reacted with an NHS ester (such as gamma-maleimidobutyric acid NHS ester (GMBS)) to form a maleimide derivative that is reactive with sulfhydryl groups. The two activated compounds (e.g. antibody and lipid) are then reacted to form a covalent linkage that is noncleavable. Periodate coupling requires the presence of oligosaccharide groups which can be present on the antibody or fragment thereof. This allows forming active aldehyde groups from the carbohydrate groups that may be present on the antibody or fragment thereof. These groups can then be reacted with amino groups on the lipid or linker generating a stable conjugate. Alternatively, the periodate oxidized antibody can be reacted with a hydrazide derivative of a lipid or linker, which will also yield a stable conjugate.

Preferably, the linker or lipid is covalently linked to a cysteine of said the antibody or fragment thereof of the invention. Preferred examples of linkers comprise Y, —(CH₂)ₙ—, —(CH₂CH₂X)ₙ—, —(CH₂CH₂CH₂X)ₙ—, —Y—(CH₂CH₂X)ₙ—, —Y—(CH₂CH₂CH₂X)ₙ, —Y—(CH₂CH₂X)ₙ—Z, —Y—(CH₂CH₂CH₂X)ₙ—Z, —Y—(CH₂CH₂X)ₙ—CH₂—Z, —Y—(CH₂CH₂CH₂X)ₙ—CH₂—Z, —Y—(CH₂CH₂X)ₙ—CH₂—CH₂—Z, —Y—(CH₂CH₂CH₂X)ₙ—CH₂—CH₂—Z, a glycosylphosphatidylinositol (GPI), a polynucleotide, an amino acid, a polypeptide, a carbohydrate and combinations thereof; wherein X is —O— or —NH—, Y is —NH—, —NH—(C═O)—, —(C═O)—NH—, —CH₂—(C═O)—NH— or —CH₂—, Z is —NH—(C═O)—, —(C═O)—, and n is an integer of 0 to 40 (i.e. 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40); more preferably n is an integer of between 4 and 24 (i.e. 4, 5, 6, 7, 8, 9, 10, 11, 12, 13, 14, 15, 16, 17, 18, 19, 20, 21, 22, 23, or 24).

If the lipid linked (optionally via said linker) to the antibody or fragment thereof of the invention is cholesterol or a derivative thereof, then it is preferred that this lipid is attached directly or via the linker to the antibody or fragment thereof through the oxygen moiety at the 3 position of the cholesterol or derivative thereof.

In a preferred embodiment the lipid, preferably cholesterol or the linker is attached to the sulphur moiety of a Cys amino acid that naturally occurs in the antibody or antibody fragment thereof or has been introduced into said antibody or fragment thereof via mutagenesis.

Particularly preferred structures of the cholesterol and the linker moiety of an antibody or fragment thereof of the invention are set out in formulas (V) to (XIV):

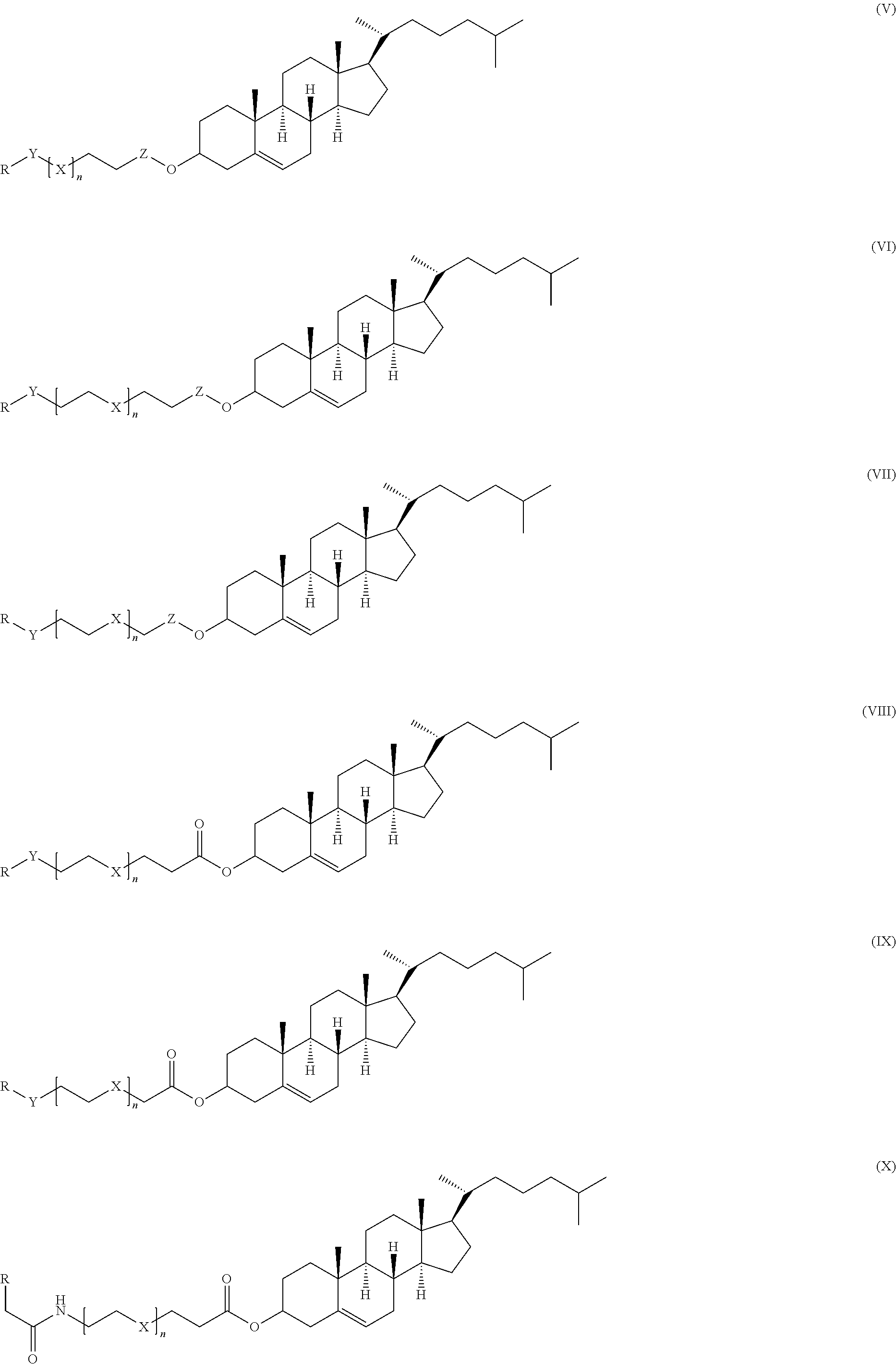

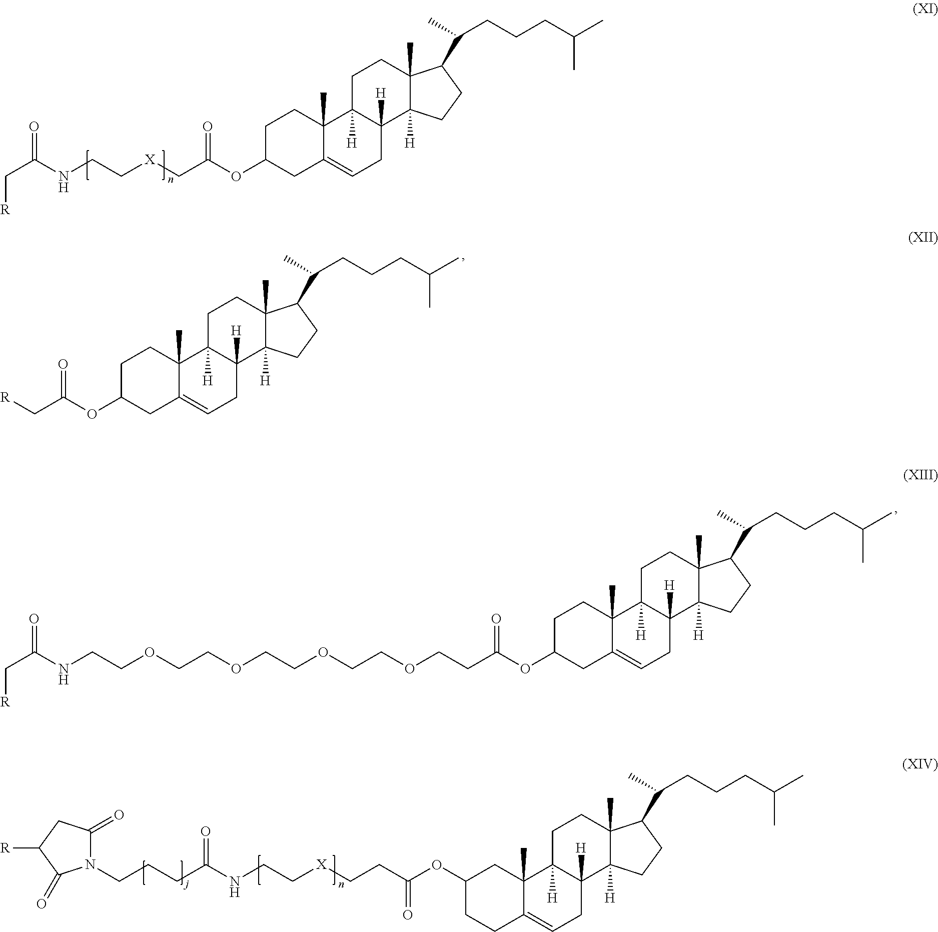

wherein X in each instance is independently selected from —NH—, —CH₂— and —O—; Y is selected from the group consisting of —CH₂—, —NH—, —NH—(C═O)—, —(C═O)—NH—, —CH₂—(C═O)—NH— and —CH₂—; Z is —NH—(C═O)—, —(C═O)—;
R designates the bond to linker or to the antibody or fragment thereof, preferably a sulphur moiety (e.g. sulfhydryl group) of an amino acid thereof; and
n is an integer of 0 to 40 (i.e. 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40) ; more preferably n is an integer of between 4 and 24 (i.e. 4, 5, 6, 7, 8, 9, 10, 11, 12, 13, 14, 15, 16, 17, 18, 19, 20, 21, 22, 23, or 24); and j is an integer selected from 0 to 40 (i.e. 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40) ; more preferably j is an integer of between 4 and 24 (i.e. 4, 5, 6, 7, 8, 9, 10, 11, 12, 13, 14, 15, 16, 17, 18, 19, 20, 21, 22, 23, or 24).

In preferred embodiments of the antibody or fragment thereof of the invention said amino acid to which said lipid or said linker is covalently linked is comprised in the light chain of said antibody or fragment thereof and most preferably is comprised in a VL domain of said antibody or fragment thereof.

In a preferred embodiment, the antibody or fragment thereof of the invention comprises a heavy chain with a VH domain and a light chain with a VL domain, wherein the VH and VL domains respectively have an amino acid sequence of any of i) through xxiv):

| | VH-domain | VL-domain |
|---|---|---|
| | (SEQ ID NO) | (SEQ ID NO): |
| i) | 2 | 3; |
| ii) | 2 | 4; |
| iii) | 6 | 7; |
| iv) | 6 | 8; |
| v) | 9 | 7 |
| vi) | 9 | 8 |
| vii) | 11 | 12; |
| viii) | 11 | 13; |
| ix) | 15 | 16; |
| x) | 15 | 17; |
| xi) | 19 | 20; |
| xii) | 19 | 21; |
| xiii) | 23 | 24; |
| xiv) | 23 | 25; |
| xv) | 27 | 28; |
| xvi) | 27 | 29; |
| xvii) | 31 | 32; |
| xviii) | 31 | 33; |
| xix) | 35 | 36; |
| xx) | 35 | 37; |
| xxi) | 39 | 40; |
| xxii) | 39 | 41; |
| xxiii) | 43 | 44; |
| xxiv) | 43 | 45 |

and wherein the light and heavy chain in total optionally comprise one, two or three single amino acid substitutions, deletions, modifications and/or insertions. It is preferred that said lipid or said liker in the aforementioned embodiments (i), (iii), (v), (ix), (xi), (xv), (xvii), (xix), (xxi), and (xxiii) is covalently linked to an amino acid, preferably cysteine, at position 20 of the respective VL domain of the antibody or fragment thereof of the invention. It is preferred that said lipid or said liker in the aforementioned embodiments (ii), (iv), (vi), (viii), (x), (xii), (xvi), (xviii), (xx), (xxii), and (xxiv) is covalently linked to an amino acid, preferably cysteine, at position 22 of the respective VL domain of the antibody or fragment thereof of the invention. It is further preferred that said lipid or said liker in the aforementioned embodiment (xiii) is covalently linked to an amino acid, preferably cysteine, at position 19 of the VL domain of the antibody or fragment thereof of the invention. It is further preferred that said lipid or said liker in the aforementioned embodiment (xiv) is covalently linked to an amino acid, preferably cysteine, at position 21 of the VL domain of the antibody or fragment thereof of the invention.

Preferably, the aforementioned antibody and fragment thereof is also capable of specifically binding to a lipid membrane such as a lipid-raft microdomain in a plasma membrane via said lipid.

In yet a further preferred embodiment of the antibody or fragment thereof according to the invention the antibody is selected from a monoclonal antibody selected from the group consisting of MAB F10, MAB CR6261, MAB D5, MAB 2F5, MAB 4E10, MAB VRC01, MAB VRC02, palivizumab, motavizumab, rituximab, trastuzumab, bevacizumab, adalimumab, cetuximab, ranibizumab, infliximab, wherein said monoclonal antibody optionally comprises one or two single amino acid substitutions, deletions, modifications and/or insertions.

As used throughout this application, the phrase “a single amino acid substitution, deletion, modification and/or insertion” of a protein or polypeptide generally refers to a modified version of the recited protein or polypeptide, e.g. one amino acid of the protein or polypeptide may be deleted, inserted, modified and/or substituted. If the polypeptide or protein comprises several single amino acid substitutions, deletions, modifications and/or insertions then the total number of such substitutions, deletions, modifications and/or insertions is indicated in each case. Said insertion is an insertion of the indicated number of single amino acids into the original polypeptide or protein. An amino acid of the protein or polypeptide may also be modified, e.g. chemically modified by the total number of modifications indicated. For example, the side chain or a free amino or carboxy-terminus of an amino acid of the protein or polypeptide may be modified by e.g. glycosylation, amidation, phosphorylation, ubiquitination, e.t.c. The chemical modification can also take place in vivo, e.g. in a host-cell, as is well known in the art. For examples, a suitable chemical modification motif, e.g. glycosylation sequence motif present in the amino acid sequence of the protein will cause the protein to be glycosylated. If the polypeptide or protein comprises one or more single amino acid substitutions, said substitutions may in each case independently be a conservative or a non-conservative substitution, preferably a conservative substitution. In a most preferred embodiment, all substitutions are of conservative nature as further defined below. In some embodiments, a substitution also includes the exchange of a naturally occurring amino acid with a not naturally occurring amino acid. A conservative substitution comprises the substitution of an amino acid with another amino acid having a chemical property similar to the amino acid that is substituted. Preferably, the conservative substitution is a substitution selected from the group consisting of:
(i) a substitution of a basic amino acid with another, different basic amino acid;
(ii) a substitution of an acidic amino acid with another, different acidic amino acid;
(iii) a substitution of an aromatic amino acid with another, different aromatic amino acid;
(iv) a substitution of a non-polar, aliphatic amino acid with another, different non-polar, aliphatic amino acid; and
(v) a substitution of a polar, uncharged amino acid with another, different polar, uncharged amino acid.

A basic amino acid is preferably selected from the group consisting of arginine, histidine, and lysine. An acidic amino acid is preferably aspartate or glutamate. An aromatic amino acid is preferably selected from the group consisting of phenylalanine, tyrosine and tryptophane. A non-polar, aliphatic amino acid is preferably selected from the group consisting of glycine, alanine, valine, leucine, methionine and isoleucine. A polar, uncharged amino acid is preferably selected from the group consisting of serine, threonine, cysteine, proline, asparagine and glutamine. In contrast to a conservative amino acid substitution, a non-conservative amino acid substitution is the exchange of one amino acid with any amino acid that does not fall under the above-outlined conservative substitutions (i) through (v).

If a protein or polypeptide comprises one or an indicated number of single amino acid deletions, then said amino acid(s) present in the reference polypeptide or protein sequence have been removed.

In a further embodiment, the CDR3 domain of the heavy chain of the antibody or fragment thereof of the invention comprises or consists of the sequence:

| | |
|---|---|
| RRGPTTXXXXXXARGPVNAMDV | (SEQ ID NO: 46) |
| or | |
| EGTTGXXXXXXPIGAFAH; | (SEQ ID NO: 47) |

wherein X may be in each instance any amino acid and wherein the lipid is covalently bound to one of the amino acids designated as X; and
wherein said sequence according to SEQ ID NO: 46 or 47 optionally comprises one single amino acid substitution, deletion, modification and/or insertion.

In yet another aspect the invention provides a pharmaceutical composition comprising the antibody or fragment thereof according to the invention and further comprising one or more pharmaceutically acceptable diluents; carriers; excipients, fillers, binders, lubricants, glidants, disintegrants, adsorbents; adjuvants and/or preservatives.

It is particularly preferred that the pharmaceutical composition of the invention can be used in the form of systemically administered medicaments. These include parenterals, which comprise among others injectables and infusions. Injectables are formulated either in the form of ampoules or as so called ready-for-use injectables, e.g. ready-to-use syringes or single-use syringes and aside from this in puncturable flasks for multiple withdrawal. The administration of injectables can be in the form of subcutaneous (s.c.), intramuscular (i.m.), intravenous (i.v.) or intracutaneous (i.c.) application. In particular, it is possible to produce the respectively suitable injection formulations as a suspension of crystals, solutions, nanoparticular or a colloid dispersed systems like, e.g. hydrosols.

Injectable formulations can further be produced as concentrates, which can be dissolved or dispersed with aqueous isotonic diluents. The infusion can also be prepared in form of isotonic solutions, fatty emulsions, liposomal formulations and micro-emulsions. Similar to injectables, infusion formulations can also be prepared in the form of concentrates for dilution. Injectable formulations can also be applied in the form of permanent infusions both in in-patient and ambulant therapy, e.g. by way of mini-pumps.

It is possible to add to parenteral drug formulations, for example, albumin, plasma, expander, surface-active substances, organic diluents, pH-influencing substances, complexing substances or polymeric substances, in particular as substances to influence the adsorption of the pharmaceutical composition of the invention to proteins or polymers or they can also be added with the aim to reduce the adsorption of the pharmaceutical composition of the invention to materials like injection instruments or packaging-materials, for example, plastic or glass.

The pharmaceutical composition of the invention can in some embodiments also be bound to microcarriers or nanoparticles in parenterals like, for example, to finely dispersed particles based on poly(meth)acrylates, polylactates, polyglycolates, polyamino acids or polyether urethanes. The pharmaceutical composition of the invention can also be modified as depot preparations, e.g. based on the “multiple unit principle”, if the composition of the invention is introduced in finely dispersed, dispersed and suspended form, respectively, or as a suspension of crystals in the medicament or based on the “single unit principle” if the composition of the invention is enclosed in a formulation, e.g. in a tablet or a rod which is subsequently implanted. These implants or depot medicaments in single unit and multiple unit formulations often consist out of so called biodegradable polymers like e.g. polyesters of lactic and glycolic acid, polyether urethanes, polyamino acids, poly(meth)acrylates or polysaccharides.

Adjuvants in a composition of the invention may preferably be aqua sterilisata (sterilized water), pH value influencing substances like, e.g. organic or inorganic acids or bases as well as salts thereof, buffering substances for adjusting pH values, substances for isotonization like e.g. sodium chloride, sodium hydrogen carbonate, glucose and fructose, tensides and surfactants, respectively, and emulsifiers like, e.g. partial esters of fatty acids of polyoxyethylene sorbitans (for example, Tween®) or, e.g. fatty acid esters of polyoxyethylenes (for example, Cremophor®), fatty oils like, e.g. peanut oil, soybean oil or castor oil, synthetic esters of fatty acids like, e.g. ethyl oleate, isopropyl myristate and neutral oil (for example, Miglyol®) as well as polymeric adjuvants like, e.g. gelatine, dextran, polyvinylpyrrolidone, additives which increase the solubility of organic solvents like, e.g. propylene glycol, ethanol, N,N-dimethylacetamide, propylene glycol or complex forming substances like, e.g. citrate and urea, preservatives like, e.g. benzoic acid hydroxypropyl ester and methyl ester, benzyl alcohol, antioxidants like e.g. sodium sulfite and stabilizers like e.g. EDTA.

When formulating the pharmaceutical composition of the present invention as suspension in a preferred embodiment thickening agents to prevent the setting of the pharmaceutical composition of the invention or, tensides and polyelectrolytes to assure the resuspendability of sediments and/or complex forming agents like, for example, EDTA are added. It is also possible to achieve complexes of the active ingredient with various polymers. Examples of such polymers are polyethylene glycol, polystyrol, carboxymethyl cellulose, Pluronics® or polyethylene glycol sorbit fatty acid ester. In particular embodiments dispersing agents can be added as further adjuvants. For the production of lyophilisates scaffolding agents like mannite, dextran, saccharose, human albumin, lactose, PVP or varieties of gelatine can be used.

In a further aspect the invention provides an antibody or fragment thereof or a pharmaceutical composition of the invention for use in the treatment or the prevention of a disease selected from the group consisting of cancer, metabolic diseases including but not limited to hyperglycemia and diabetes, obesity, hypertension, hypercholesterolemia, allergy, asthma, Alzheimer's disease, and infectious diseases including but not limited to a disease caused by a virus, a bacterium and a fungus. Preferably, the disease caused by a virus is caused by a virus selected from the group consisting of HIV, Influenza virus, Hepatitis B virus, Hepatitis C virus, Rhinovirus, Herpes virus, Herpes simplex virus, West Nile Virus, Dengue virus, SARS-CoV, Varicella-zoster virus, Pseudorabies virus, Vesicular stomatits virus, Borna disease virus, Newcastle disease virus, Vaccinia virus, Rotavirus, Sendai virus, Measles virus, Mumps virus, Human Parainfluenza virus, Respiratory syncytical virus, Hendra virus, Nipah virus, Ebola virus, Marburg virus, Junin virus, Machupo virus, Guanairito virus and Lassa virus.

Certain amounts of the antibody, fragment thereof and pharmaceutical composition according to the invention are preferred for the therapy of a disease, e.g. between 5 and 400 mg more preferably between 10 and 375 mg and most preferably between 20 and 100 mg of antibody or fragment thereof per m² body surface of the patient. It is, however, understood that depending on the severity of the disease, the type of the disease, as well as on the respective patient to be treated, e.g. the general health status of the patient, etc., different doses of the pharmaceutical composition according to the invention are required to elicit a therapeutic effect. If a well known and well characterized antibody is conjugated to a lipid according to the invention, it is preferred that the modified antibody according to the invention is administered at a dosage that is between ⅓ to ⅔ lower than the dosage recommended for the original, unmodified antibody. The determination of the appropriate dose lies within the discretion of the attending physician.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following Figures are merely illustrative of the present invention and should not be construed to limit the scope of the invention as indicated by the appended claims in any way.

### BRIEF DESCRIPTION OF THE FIGURES

FIG. 1. SITES OF CHOLESTEROL ATTACHMENT FOR MAB D5. The figure is based on the crystal structure of the complex of Fab D5 with 5-helix, as reported in: Luftig et al., Nat. Struct. Mol. Biol. 13 (2006) 740-746. The residues chosen for cholesterol attachment (T²⁰, T²² of VL) are depicted as spheres (A) Side view. Approximately perpendicular to the membrane plane. (B) Top view, looking from the target cell membrane into the viral membrane. The Fab is rotated 90°, to show the absence of steric hindrance between the cholesterol attachment site(s) and the antigen-binding surface of D5.

FIG. 2. SITES OF CHOLESTEROL ATTACHMENT FOR MAB 2F5. The figure is based on the crystal structure of the complex of Fab 2F5 with a peptide corresponding to its epitope on gp41, as reported in Ofek, G., et al., 2010; Relationship between Antibody 2F5 Neutralization of HIV-1 and Hydrophobicity of Its Heavy Chain Third Complementarity-Determining Region. J Virol 84:2955-2962; and in Ofek, G., et al., 2004; Structure and mechanistic analysis of the anti-human immunodeficiency virus type 1 antibody 2F5 in complex with its gp41 epitope. J Virol 78:10724-37). The residues chosen for cholesterol attachment (T²⁰, T²² of VL) are depicted as spheres. The linker and cholesterol conjugated to Fab 2F5, the rest of the extracellular domain and the transmembrane domain of gp41 are in cartoon representation, to show their approximate geometry with respect to the plane of the membrane.

FIG. 3. SITES OF CHOLESTEROL ATTACHMENT FOR MAB 4E10. The figure is based on the crystal structure of the complex of Fab 4E10 with a peptide corresponding to its epitope on gp41, as reported in Cardoso, R. M. F., et al., 2005; Broadly Neutralizing Anti-HIV Antibody 4E10 Recognizes a Helical Conformation of a Highly Conserved Fusion-Associated Motif in gp41. Immunity 22:163-173. The residues chosen for cholesterol attachment (T²⁰, S²² of V_{L}) are depicted as spheres. The linker and cholesterol conjugated to Fab 4E10, the rest of the extracellular domain and the transmembrane domain of gp41 are in cartoon representation, to show their approximate geometry with respect to the plane of the membrane.

FIG. 4. SITES OF CHOLESTEROL ATTACHMENT FOR MAB VRC01. The figure is based on the crystal structure of the complex of Fab VRC01 with gp120, as reported in Zhu, T., et al., 2010; Structural Basis for Broad and Potent Neutralization of HIV-1 by Antibody VRC01. Science July 2010, DOI: 10.1126/science.1192819. The residues chosen for cholesterol attachment (I²⁰, S²² of VL) are depicted as spheres. The linker and cholesterol conjugated to Fab VRC01, and the rest of gp120 are in cartoon representation, to show their approximate geometry with respect to the plane of the membrane.

FIG. 5. SITES OF CHOLESTEROL ATTACHMENT FOR MAB CR6261. The figure is based on the crystal structure of the complex of Fab CR6261 with the hemagglutinin (HA) of an H5N1 influenza virus, as reported in Ekiert, D. C., et al., 2009; Antibody recognition of a highly conserved influenza virus epitope, Science, 324:246-51. The residues chosen for cholesterol attachment (T¹⁹, S²¹ of VL) are depicted as spheres. The linker and cholesterol conjugated to Fab CR6261, the C-terminal sequence of HA and its transmembrane domain are in cartoon representation, to show their approximate geometry with respect to the plane of the membrane.

FIG. 6. SITES OF CHOLESTEROL ATTACHMENT FOR RITUXIMAB. The figure is based on the crystal structure of the complex of the Rituximab Fab with a peptide corresponding to its epitope in the extracellular domain of CD20, as reported in Du, J. et al., 2007; Structural basis for recognition of CD20 by therapeutic antibody Rituximab, J. Biol. Chem., 282:15073-15080. The residues chosen for cholesterol attachment (T²⁰, S²² of VL) are depicted as spheres. The linker and cholesterol conjugated to the Rituximab Fab and the transmembrane domain of CD20 are in cartoon representation, to show their approximate geometry with respect to the plane of the membrane

FIG. 7. SITES OF CHOLESTEROL ATTACHMENT FOR TRASTUZUMAB. The figure is based on the crystal structure of the complex of the Trastuzumab Fab with the juxtamembrane region of the extracellular domain of HER2, as reported in Cho H.-S. et al., 2004; Structure of the extracellular region of HER2 alone and in complex with the Herceptin Fab, Nature, 421:756-60. The residues chosen for cholesterol attachment (T²⁰, T²² of VL) are depicted as spheres. The linker and cholesterol conjugated to the Trastuzumab Fab, the C-terminal sequence of HER2 and its transmembrane domain are in cartoon representation, to show their approximate geometry with respect to the plane of the membrane.

FIG. 8. SITES OF CHOLESTEROL ATTACHMENT FOR CETUXIMAB. The figure is based on the crystal structure of the complex of the Cetuximab Fab with the extracellular domain of EGFR, as reported in Li, S. et al., 2005; Structural basis for inhibition of the Epidermal Growth Factor receptor by cetuximab, Cancer Cell, 7:301-311. The residues chosen for cholesterol attachment (S²⁰, S²² of VL) are depicted as spheres. The linker and cholesterol conjugated to the Cetuximab Fab, the C-terminal sequence of EGFR and its transmembrane domain are in cartoon representation, to show their approximate geometry with respect to the plane of the membrane. Comparison with the structure of the Trastuzumab-HER2 complex (FIG. 5) illustrates the requirement for a different length of the linker.

FIG. 9. Illustrates the preferred and optimized locations of cysteine amino acids in the Fab domain of MAB D5, that are ideally positioned for covalent linkage to a lipid or a linker including a lipid according to the invention. (*) marks introduced cysteine amino acids.

FIG. 10. Illustrates the preferred and optimized locations of cysteine amino acids in the Fab domain of MAB 2F5, that are ideally positioned for covalent linkage to a lipid or a linker including a lipid according to the invention. Also shown is a double mutant of Fab 2F5 with no antiviral activity. (*) marks introduced cysteine amino acids.

FIG. 11. Illustrates the preferred and optimized locations of cysteine amino acids in the Fab domain of MAB 4E10, that are ideally positioned for covalent linkage to a lipid or a linker including a lipid according to the invention. (*) marks introduced cysteine amino acids.

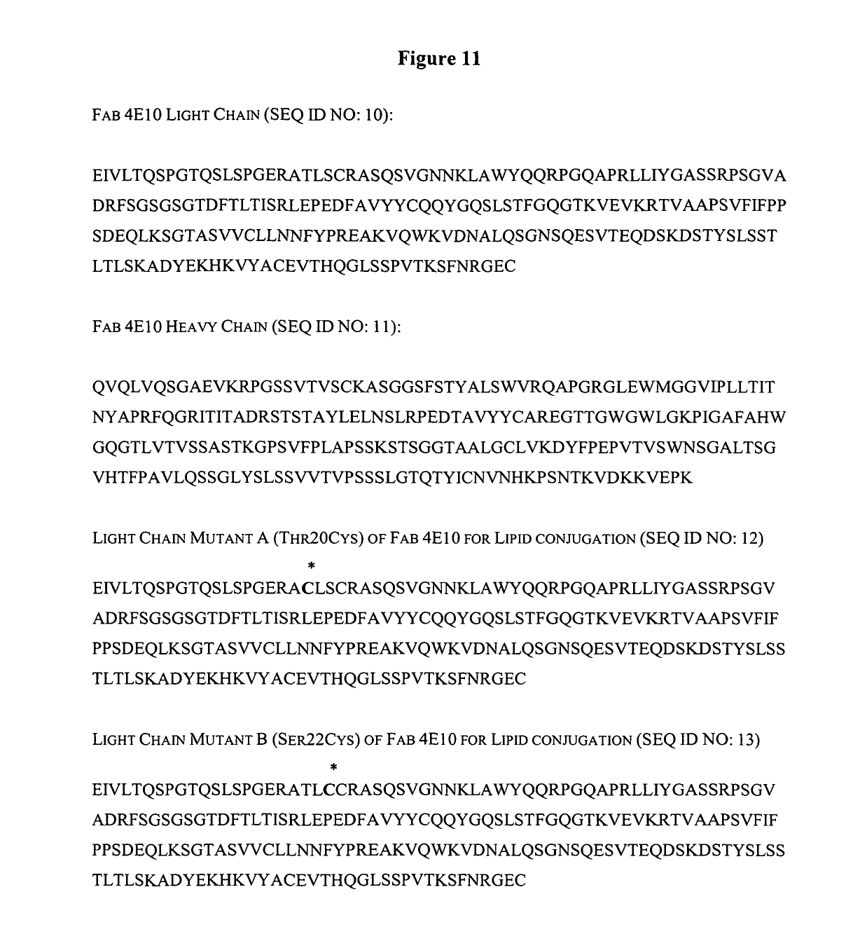

FIG. 12. Illustrates the preferred and optimized locations of cysteine amino acids in the Fab domain of MAB VRC01, that are ideally positioned for covalent linkage to a lipid or a linker including a lipid according to the invention. (*) marks introduced cysteine amino acids.

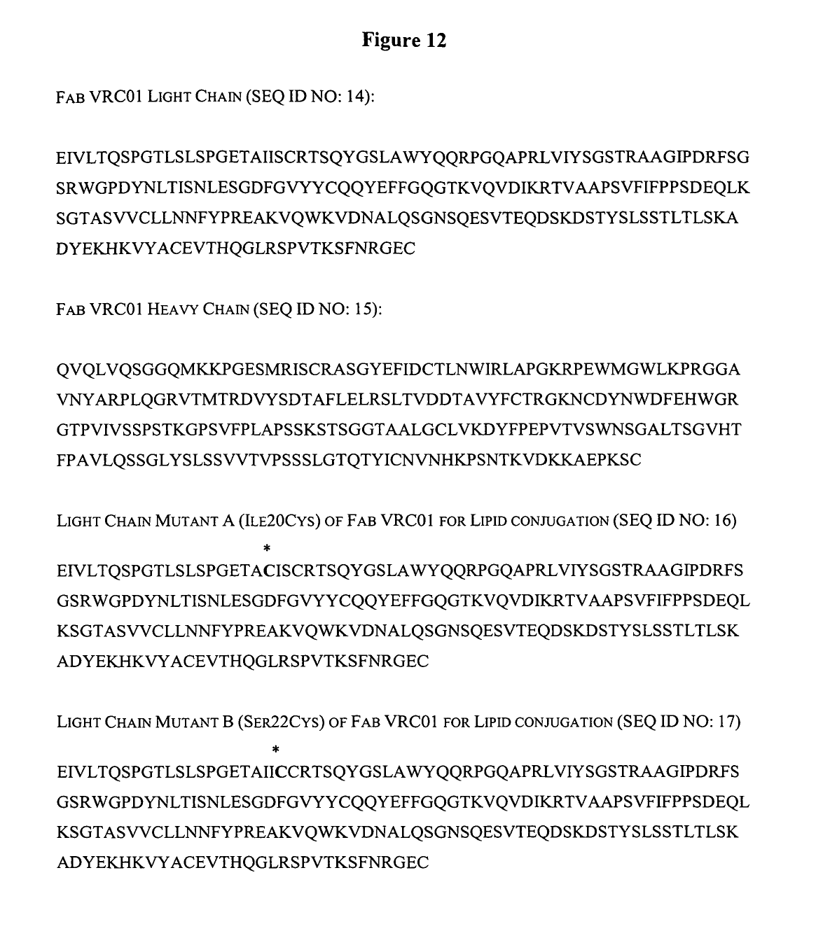

FIG. 13. Illustrates the preferred and optimized locations of cysteine amino acids in the Fab domain of MAB VRC02, that are ideally positioned for covalent linkage to a lipid or a linker including a lipid according to the invention. (*) marks introduced cysteine amino acids.

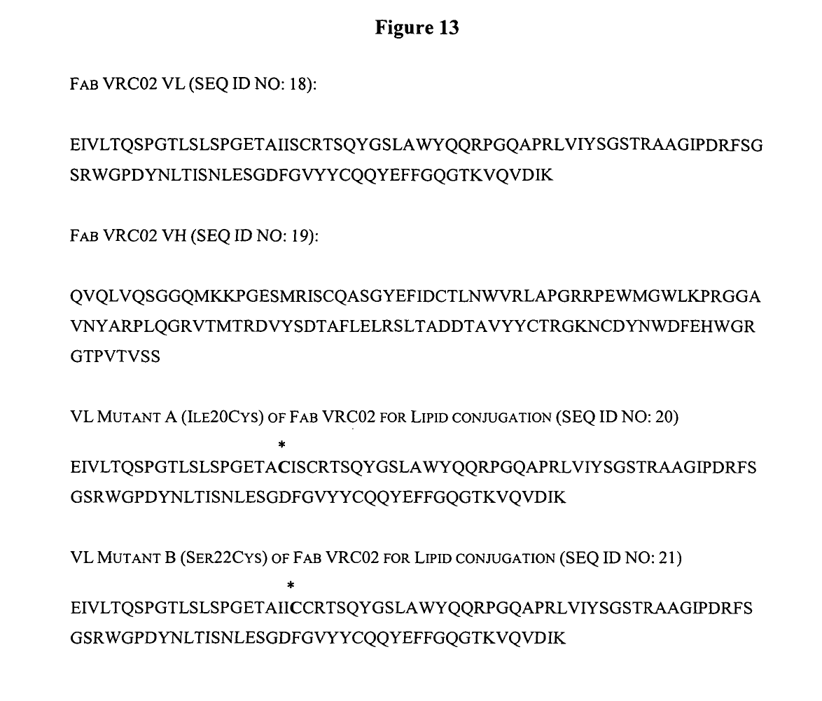

FIG. 14. Illustrates the preferred and optimized locations of cysteine amino acids in the Fab domain of mAb CR6261, that are ideally positioned for covalent linkage to a lipid or a linker including a lipid according to the invention. (*) marks introduced cysteine amino acids.

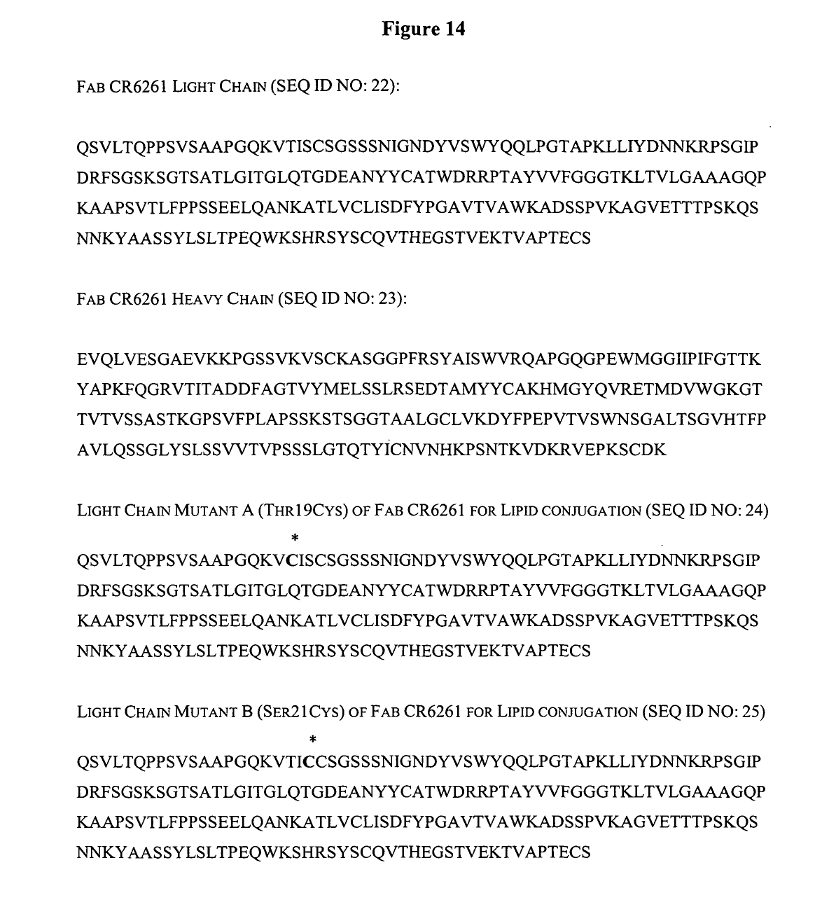

FIG. 15. Illustrates the preferred and optimized locations of cysteine amino acids in the Fab domain of Palivizumab, that are ideally positioned for covalent linkage to a lipid or a linker including a lipid according to the invention. (*) marks introduced cysteine amino acids.

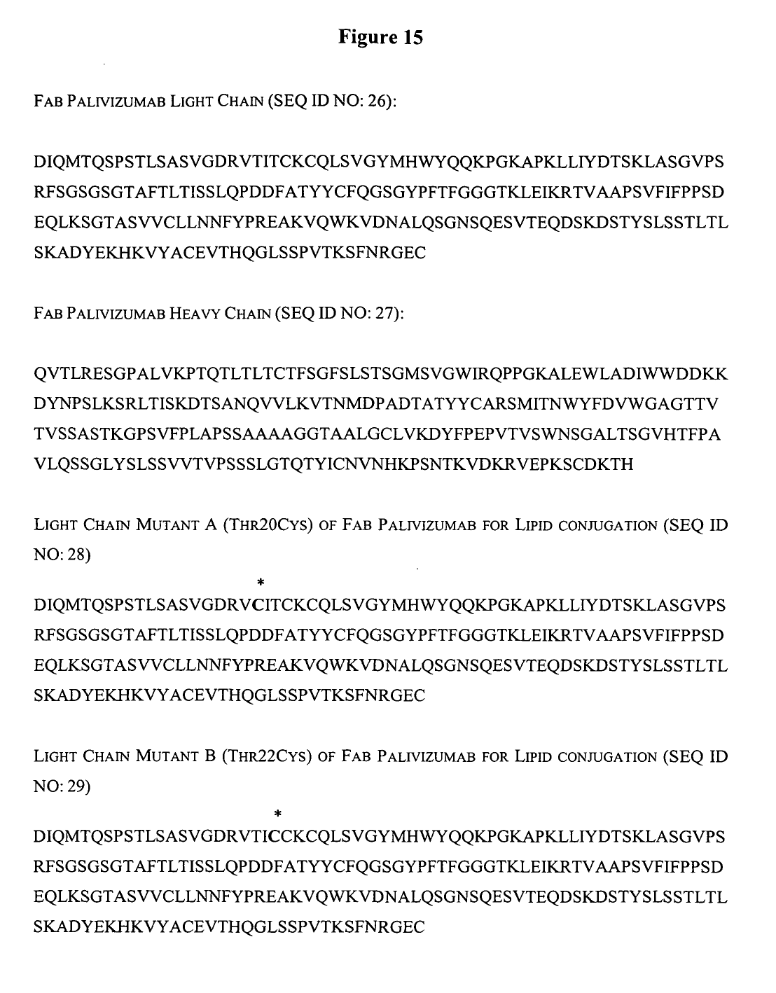

FIG. 16. Illustrates the preferred and optimized locations of cysteine amino acids in the Fab domain of Motavizumab, that are ideally positioned for covalent linkage to a lipid or a linker including a lipid according to the invention. (*) marks introduced cysteine amino acids.

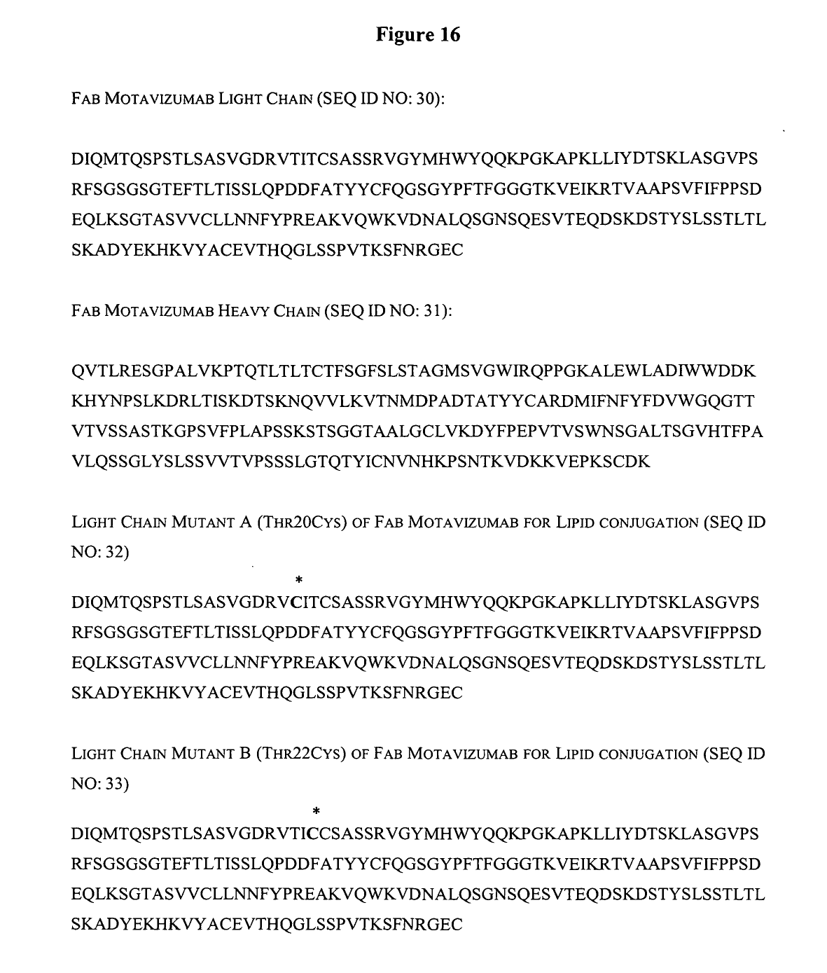

FIG. 17. Illustrates the preferred and optimized locations of cysteine amino acids in the Fab domain of Rituximab, that are ideally positioned for covalent linkage to a lipid or a linker including a lipid according to the invention. (*) marks introduced cysteine amino acids

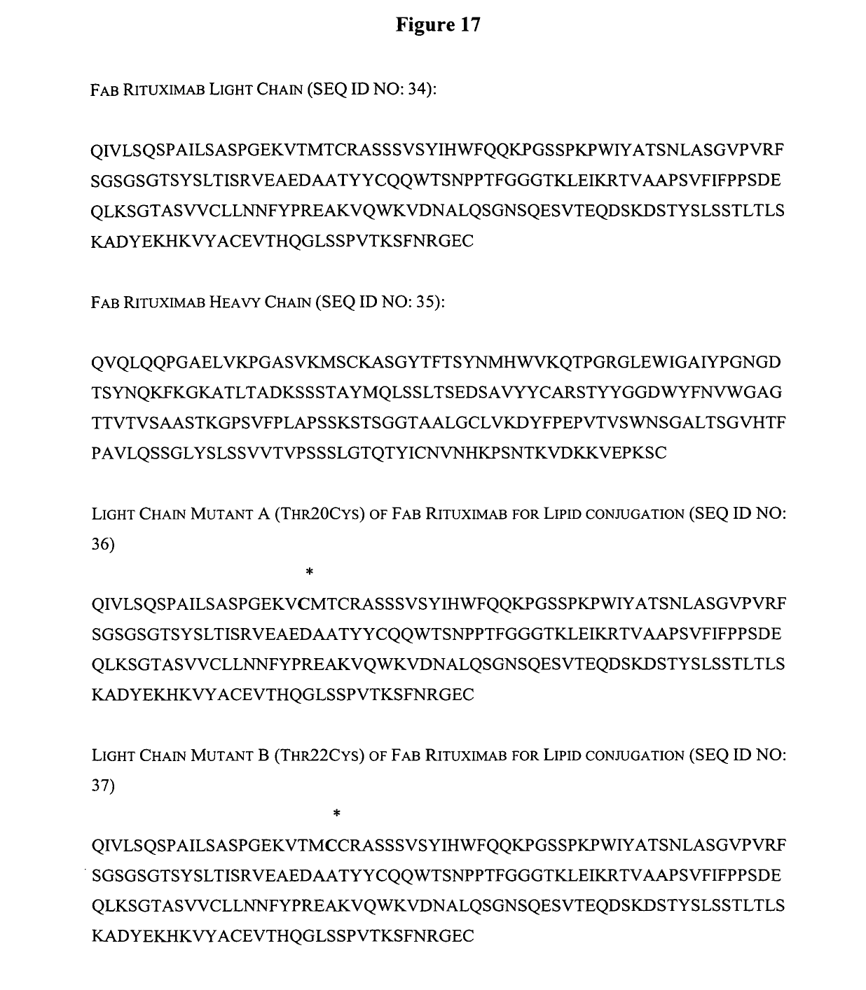

FIG. 18. Illustrates the preferred and optimized locations of cysteine amino acids in the Fab domain of Trastuzumab, that are ideally positioned for covalent linkage to a lipid or a linker including a lipid according to the invention. (*) marks introduced cysteine amino acids.

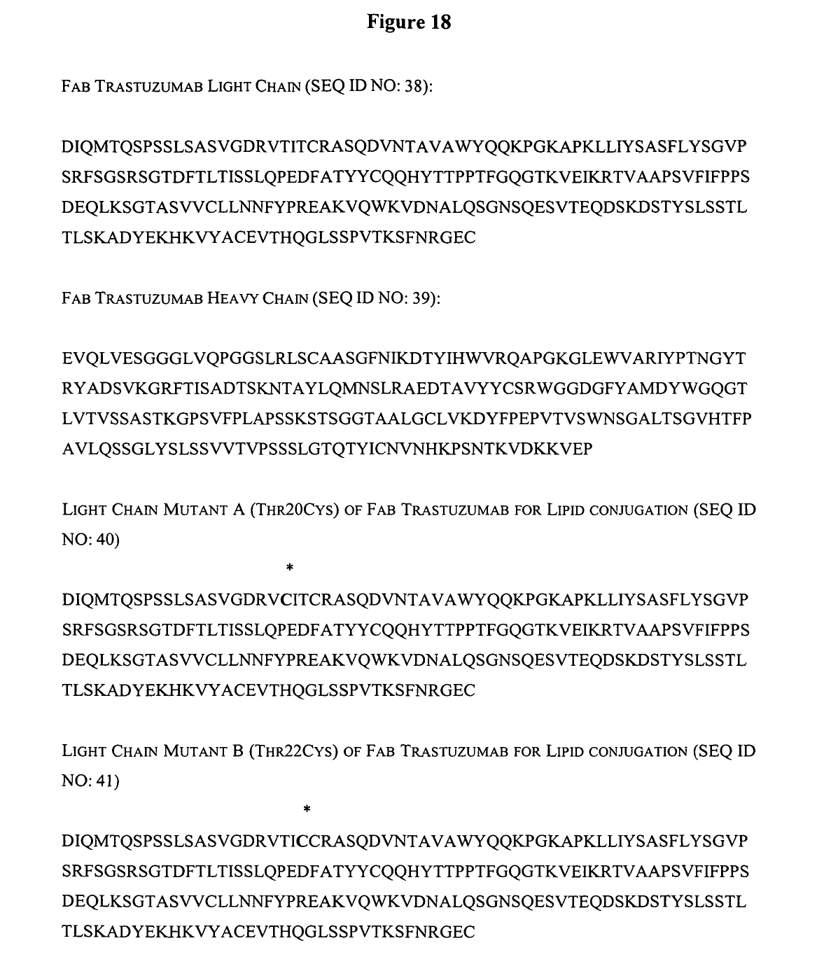

FIG. 19. Illustrates the preferred and optimized locations of cysteine amino acids in the Fab domain of Cetuximab, that are ideally positioned for covalent linkage to a lipid or a linker including a lipid according to the invention. (*) marks introduced cysteine amino acids.

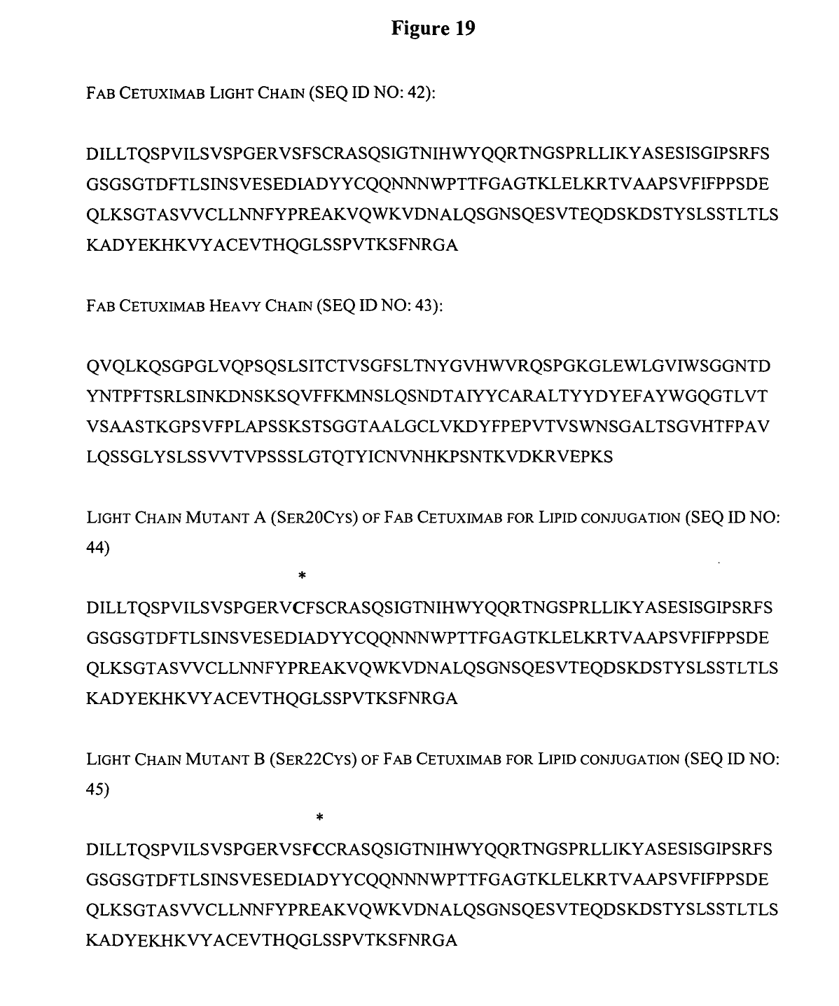

FIG. 20. Illustrates the amino acid sequences of the heavy chain and the light chain of Trastuzumab and the location of the introduced cysteine amino acid in the light chain mutant A (Thr20Cys) of Trastuzumab (TrastuzumabC20; HerceptinC20). (*) marks introduced cysteine amino acid.

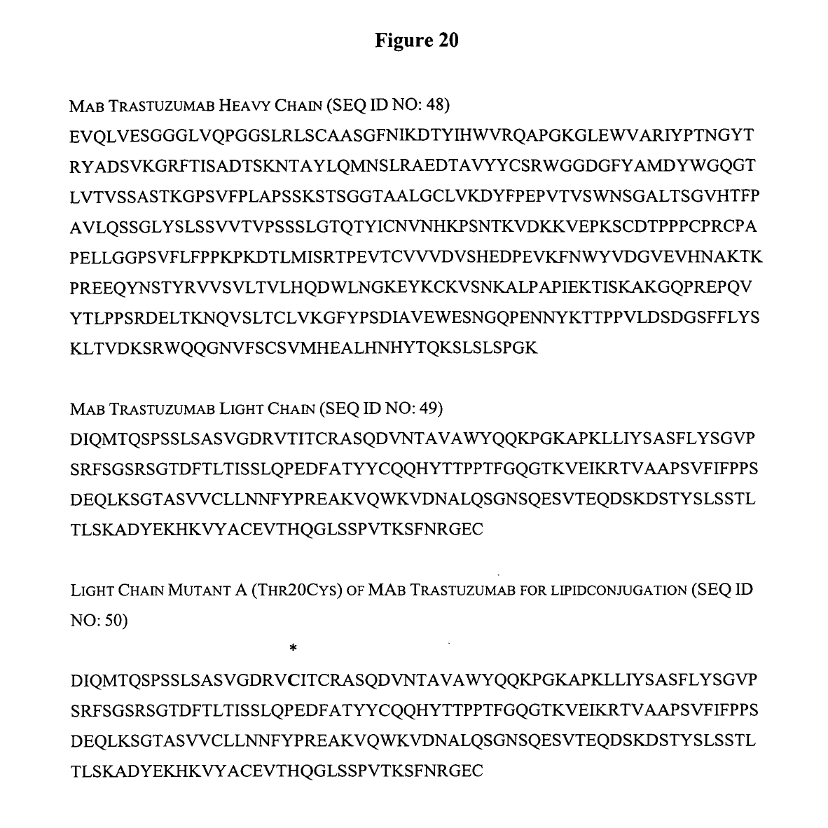

FIG. 21. Reconstructed MS spectrum of the light chain of the TrastuzumabC20 mAb after reaction with Cholesterol-PEPG₁₂-Maleimide. The two peaks at mass=23,436 and mass=24,589 correspond to the unconjugated and conjugated light chain after reduction and alkylation, respectively (calculated difference in mass: 1151.5, found: 1153).

FIG. 22. Cell-ELISA with unconjugated (TrastuzumabC20, HerceptinC20) and cholesterol-conjugated (TrastuzumabC20-CHOL, HerceptinC20-CHOL) mAb on ErbB2-positive SKBR3 cells

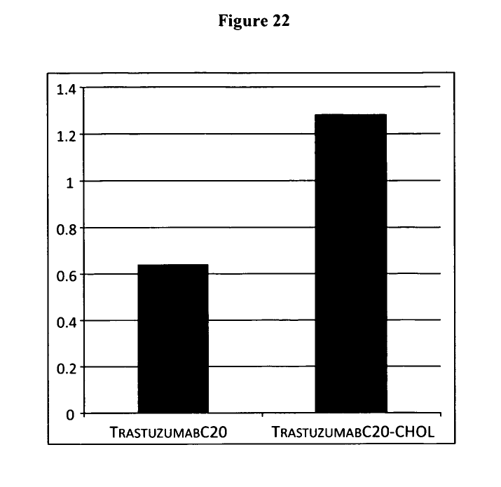

### EXPERIMENTAL DETAILS

### EXAMPLE 1

Lipid-linked Antibody Synthesis. Methods of making antibodies comprising naturally and non-naturally occurring amino acids are well known in the art. Synthetic or microbiological methods can be used. Free cysteines introduced into antibodies offer the possibility to be conjugated with a lipid or linker according to the invention. Thiol-reactive chemistry is also very convenient for antibody derivatization, since most antibodies lack cysteines, save those involved in inter- and intra-chain disulfide bonds. Several authors have shown that it is possible to engineer unpaired cysteines in antibodies, and use them for regioselective conjugation of biotin and cytotoxic drugs for targeted therapy (31, 32, 43, 70, 74). As an example of thiol-reactive chemistry, conjugation to cholesterol can be accomplished though reaction of a bromoacetyl cholesterol derivative with a free cysteine residue in the antibody. Although very convenient and utilized for the examples below, thiol-reactive chemistry should not be taken as the only possible way to attach a lipid at selected locations in the antibody.

### EXAMPLE 2

General scheme for the synthesis of cholesterol-derivatized antibody or derivative thereof. The cholesterol moiety is attached to the antibody via a thioether linkage with the thiol group of cysteine residue in the antibody. The conjugate is prepared via chemoselective reaction between a bromoacetyl group (on cholesterol) and a free thiol (on the antibody), as described in Zeng et al, Vaccine, 2001, 19, 3843-3852.

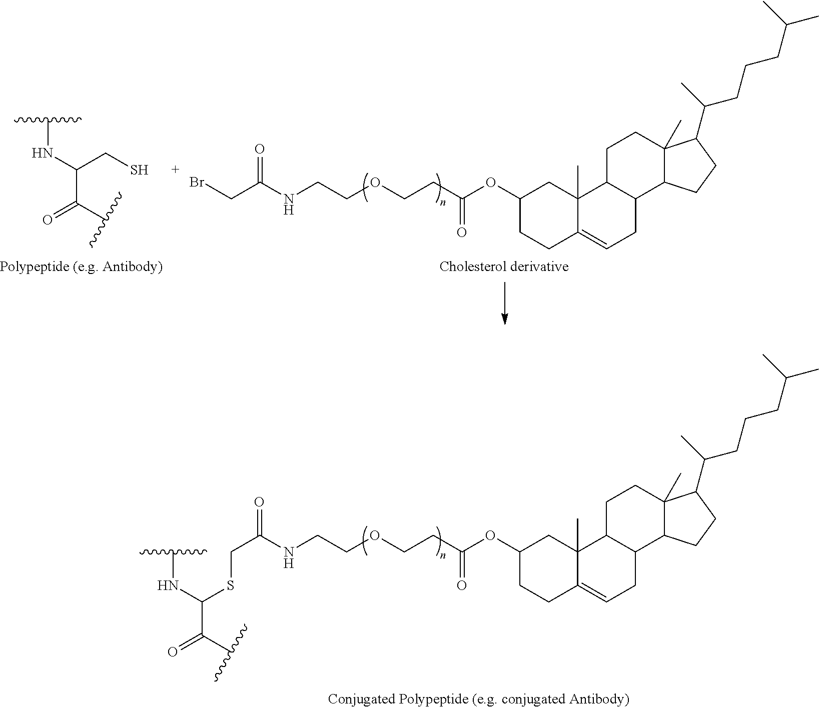

Alternatively, the conjugate is prepared via reaction between a maleimide group (on cholesterol) and a free thiol (on the antibody).

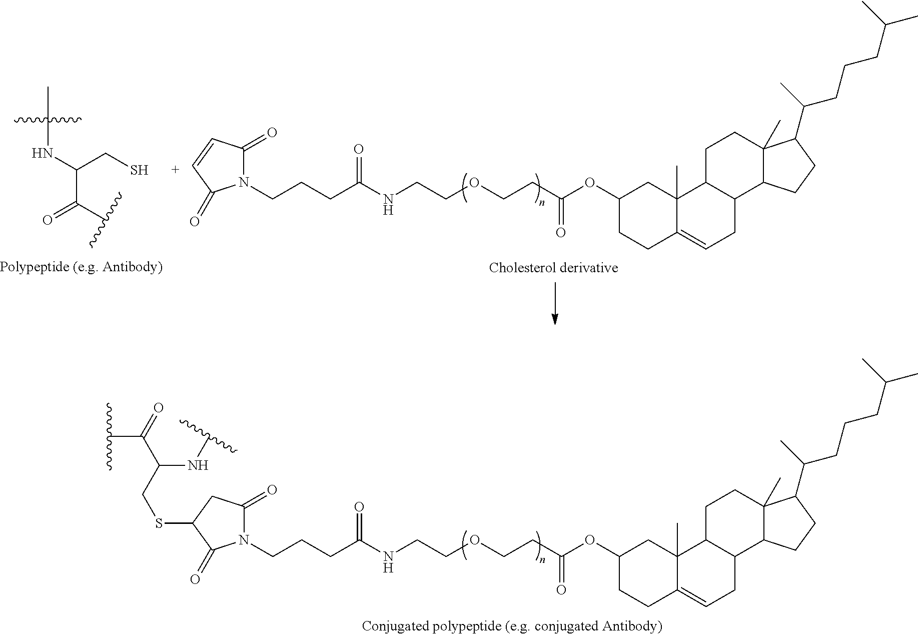

The required cholesterol derivatives bearing a bromoacetyl or a maleimide group can be made as described in the Examples, or by analogy, thereto, by using commercially available compounds or by well known methods. Derivatives of cholesterol are commercially available or can be made from commercially available materials by well known methods.

### EXAMPLE 3

### Synthesis of Bromoacetyl-Cholesterol

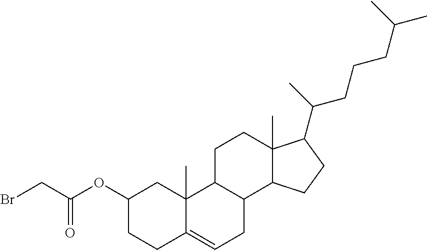 [SMILES: CC(C)CCCC(C)C1CCC2C3CC=C4CCC(OC(=O)CBr)CC4(C)C3CCC12C]

A mixture of 100 mg of cholesterol and 40 mg of bromoacetic acid (1.1 eq) was dissolved in 10 mL of anhydrous dichloromethane. Then 44 μl (1.1 eq) of DIPC (N,N-diisopropylcarbodiimide) and 1.5 mg (0.05 eq) of DMAP (4-dimethylaminopyridine) were added. The solution was left stirring at room temperature for 48 h and analyzed by TLC using a mixture of n-hexane/EtOAc 10/1 as solvent systems. The solvent was evaporated and the reaction product was purified by silica gel flash chromatography in n-hexane/dichloromethane 1/1. The fractions containing the product were pooled, evaporated and then lyophilized in water/acetonitrile 20/80. The purified product was analyzed by NMR. Yield: 73%.

### EXAMPLE 4

### Synthesis of Bromoacetyl-PEG₄-Cholesterol

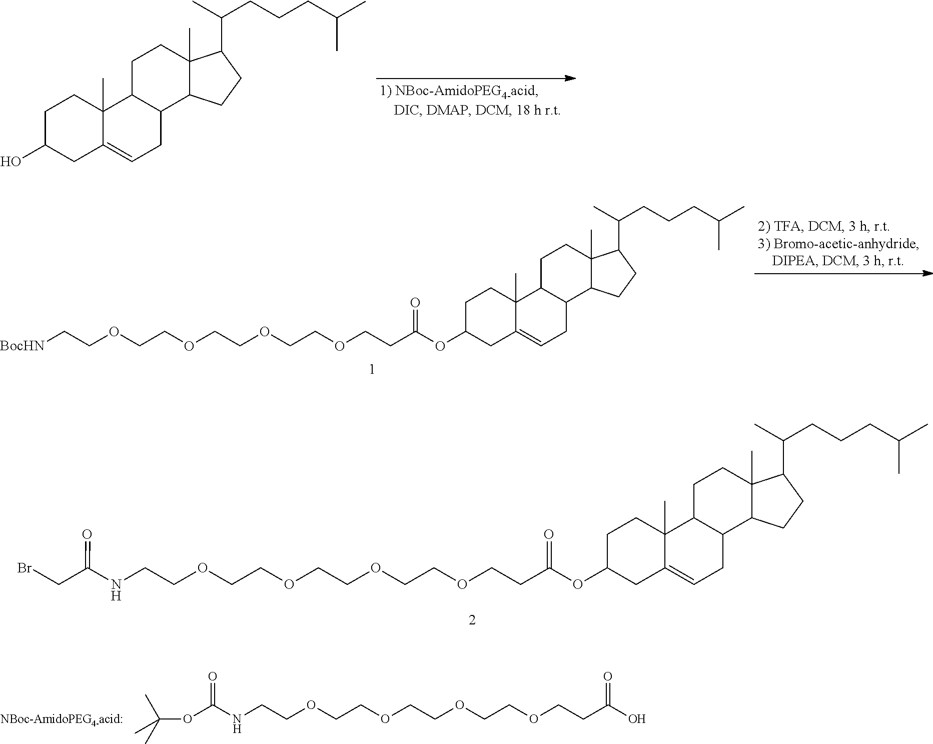

4.1 Cholest-5-en-3-yl-2,2-dimethyl-4-oxo-3,8,11,14,17-pentaoxa-5-azaicosan-20-oate (1): N-t-boc-amido-dPEG₄™ acid (1 g, 2.7 mmol, Product N° 10220, Quanta BioDesign, Ltd.) was added to a solution of cholesterol (0.99 g, 2.7 mmol) in 40 mL of CH₂Cl₂, followed by N,N′-diisopropylcarbodiimide (0.43 mL, 3.2 mmol) and 4-dimethylamino-pyridine (16 mg, 5%). The mixture was stirred at room temperature overnight and the solvent was evaporated under vacuo. The crude was dissolved in EtOAc, washed with HCl 1N, saturated NH₄Cl and brine, dried over Na₂SO₄, filtered and concentrated. The crude was purified by flash column chromatography (BIOTAGE) on silica gel with a gradient 25-50% EtOAc in petroleum ether to afford 1.48 g of desired compound as incolor oil (Yield 75%).

4.2 Cholest-5-en-3-yl 1-bromo-2-oxo-6,9,12,15-tetraoxa-3-azaoctadecan-18-oate (2): Trifluoroacetic acid (2 mL, 26 mmol) was added to a solution of 1 (1.48 g, 2 mmol) in 10 ml of CH₂Cl₂ and the mixture was stirred at room temperature for 3 h. All the volatiles were removed under vacuo and the crude was lyophilized to obtain an incolor oil that was dissolved in 60 mL of CH₂Cl₂. Bromoacetic anhydride (0.62 g, 2.4 mmol) was added followed by N,N-diisopropylethylamine (0.65 mL, 3.7 mmol) and the mixture was stirred at room temperature for 3 h. The solvent was removed under vacuo and the crude purified by flash column chromatography on silica gel (BIOTAGE) with a gradient 50-90% of EtOAc in petroleum ether to obtain 1.1 g of desired compound as a colourless oil with a yield of 74% in two steps.

### EXAMPLE 5

### Synthesis of Bromoacetyl-PEG₁₂-Cholesterol

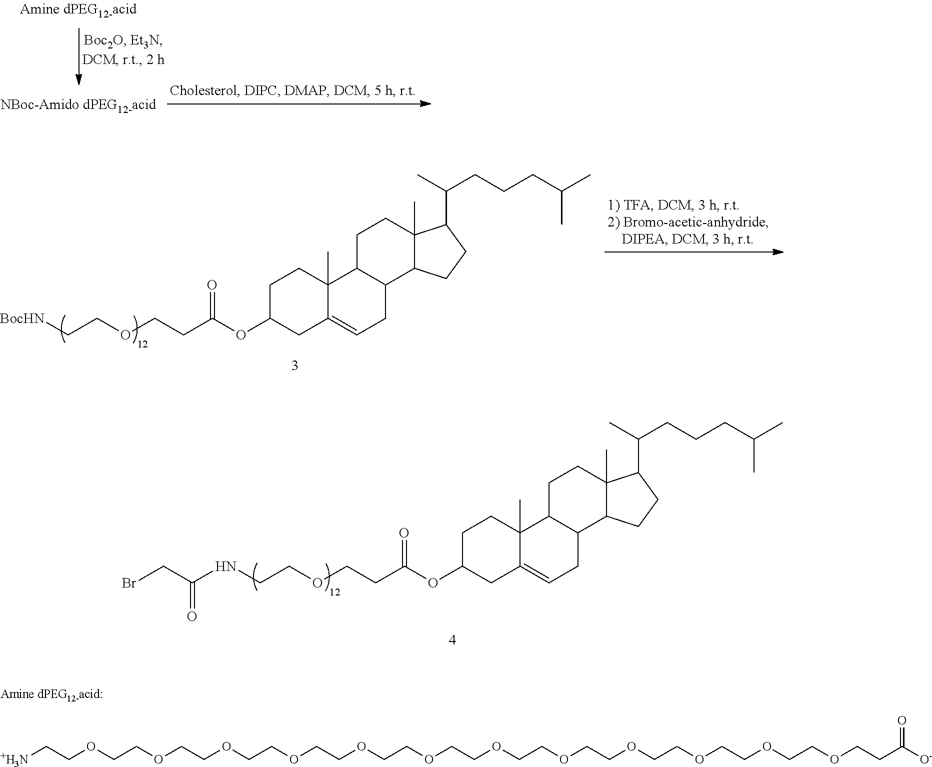

5.1 Cholest-5-en-3-yl-2,2-dimethyl-4-oxo-3,8,11,14,17,20,23,26,29,32,35,38,41-tridecaoxa-5-azatetratetracontan-44-oate (3)

Amine-dPEG₁₂™ acid (1.65 g, 2.7 mmol, Product N° 10287, Quanta BioDesign, Ltd.) was dissolved in 15 mL of dichloromethane and Boc-anhydride (0.7 g, 3.2 mmol) was added followed by triethyl amine (0.75 ml, 5.4 mmol). The mixture was stirred at room temperature for 2h and then the solvent was evaporated under reduced pressure.

Crude N-Boc Amido-dPEG₁₂™ acid was added to a solution of cholesterol (1.24g, 3.2 mmol) in 55 mL of CH₂Cl₂, followed by N,N′-diisopropylcarbodiimide (0.62 mL, 4 mmol) and 4-dimethylamino-pyridine (16 mg, 5%). The mixture was stirred at room temperature for 4 hours and the solvent was evaporated under vacuum. The crude was purified by flash column chromatography (BIOTAGE) on silica gel with a gradient 2-5% MeOH in dichloromethane to afford 1.57 g of desired compound as incolor oil (Yield 55% in two steps).

### 5.2 Cholest-5-en-3-yl-1-bromo-2-oxo-6,9,12,15,18,21,24,27,30,33,36,39-dodecaoxa-3-azadotetracontan-42-oate (4)

Trifluoroacetic acid (1.7 mL, 22 mmol) was added to a solution of 3 (1.57 g, 1.5 mmol) in 8.5 ml of CH₂Cl₂ and the mixture was stirred at room temperature for 3h until disappearance of starting material. All the volatiles were removed under vacuum and the crude was lyophilized to obtain an incolor oil that was dissolved in 45 mL of CH₂Cl₂. Bromoacetic anhydride (0.48 g, 1.8 mmol) was added followed by N,N-diisopropylethylamine (0.52 mL, 3 mmol) and the mixture was stirred at room temperature for 4 h. The solvent was removed under vacuum and the crude purified by flash column chromatography on silica gel (BIOTAGE) with a gradient 2-4% MeOH in dichloromethane to afford 1.11 g of desired compound as incolor oil (Yield 67% in two steps).

### EXAMPLE 6

### Synthesis of Cholesterol-Linked Antibodies

The antibody is prepared by conjugation between bromoacetyl-cholesterol and the antibody. The cholesterol derivative is incubated with an antibody at a molar ratio 10:1, for 3-12 h at room temperature. To ensure reactivity of the thiol group of the antibody not engaged in a disulfide bond, prior to conjugation the antibody is treated with a mild reducing agent, such as Tris-2-carboxyethyl-phospine hydrochloride (TCEP) or free cysteine. The cholesterol-antibody product is purified on a HiTrap S column (GE Helthcare Biosciences) to remove excess reagents. The conjugated antibody is buffer-exchanged in 50 mM phosphate buffer pH 7, and concentrated to approximately 20 mg/mL on spin filters with a molecular weight cutoff of 30 kDa. Alternatively the cholesterol-derivatized antibody can be purified via a protein-A or protein-G Agarose columns as is well known in the art.

### EXAMPLE 7

### Synthesis of PEG₄-Cholesterol-Conjugated Antibodies

The antibody is prepared by conjugation between bromoacetyl-PEG₄-cholesterol and the antibody. The PEG₄-cholesterol derivative is incubated with an antibody at a molar ratio 10:1, for 3-12 h at room temperature. To ensure reactivity of the thiol group of the antibody not engaged in a disulfide bond, prior to conjugation the antibody is treated with a mild reducing agent, such as Tris-2-carboxyethyl-phospine hydrochloride (TCEP) or free cysteine. The cholesterol-antibody product is purified on a HiTrap S column (GE Helthcare Biosciences) to remove excess reagents. The conjugated antibody is buffer-exchanged in 50 mM phosphate buffer pH 7, and concentrated to approximately 20 mg/mL on spin filters with a molecular weight cutoff of 30 kDa. Alternatively the cholesterol-derivatized antibody can be purified via a protein-A or protein-G Agarose columns as is well known in the art.

### EXAMPLE 8

### Synthesis of PEG₁₂-Cholesterol-Conjugated Antibodies

The antibody is prepared by conjugation between bromoacetyl-PEG₁₂-cholesterol and the antibody. The PEG₁₂-cholesterol derivative is incubated with an antibody at a molar ratio 10:1, for 3-12 h at room temperature. To ensure reactivity of the thiol group of the antibody not engaged in a disulfide bond, prior to conjugation the antibody is treated with a mild reducing agent, such as Tris-2-carboxyethyl-phospine hydrochloride (TCEP) or free cysteine.

The cholesterol-antibody product is purified on a HiTrap S column (GE Helthcare Biosciences) to remove excess reagents. The conjugated antibody is buffer-exchanged in 50 mM phosphate buffer pH 7, and concentrated to approximately 20 mg/mL on spin filters with a molecular weight cutoff of 30 kDa. Alternatively the cholesterol-derivatized antibody can be purified via a protein-A or protein-G Agarose columns as is well known in the art.

### EXAMPLE 9

### Selection of Preferred Conjugation Locations within the Antibody

Among the many possible locations for the introduction of e.g. a reactive cysteine, preferably an amino acid of the antibody is chosen for conjugation, which will allow the interaction of the conjugated lipid moiety with a lipid raft of the target membrane, however without perturbing the ability of the antibody to specifically bind its protein epitope.

In the following, a number of examples are given of cholesterol conjugations according to the invention to known antiviral antibodies. Unexpectedly, for all the antibodies a specific location of the light chain, residues 19 or 20 and 21 or 22 of the light chain, represent alternative, optimal sites of attachment for cholesterol. These positions are class I variants according to the definition of Voynov et al. (Voynov, V., N. Chennamsetty, V. Kayser, H. J. Wallny, B. Helk, and B. L. Trout. 2010. Design and application of antibody cysteine variants. Bioconjug Chem 21:385-92), i.e. they yield conjugates with a small fluorophore that are labeled exclusively at the engineered cysteine, and remain monomeric and stable after conjugation. Conjugation of cholesterol at either residue yield antibodies with excellent pharmaceutical properties and considerably improved antiviral potency.

### 1. Cholesterol Derivative of the HIV mAb D5.

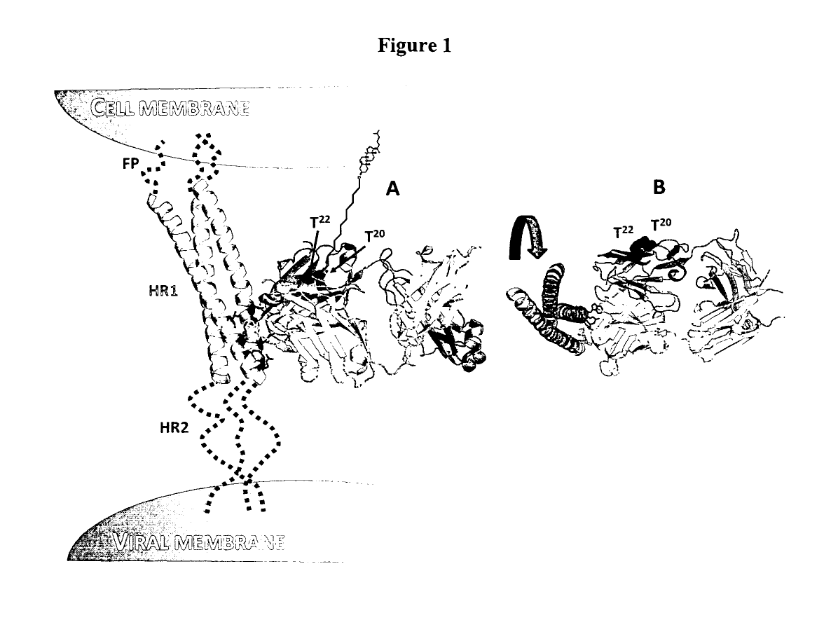

FIG. 1 shows the position of Thr²⁰ and Thr²² of the light chain in the crystal structure of the complex of mAb D5 with its peptide epitope (Thr²⁰ and Thr²² represented as spheres). The complex is oriented relative to the viral membrane according to the currently accepted model (Luftig, M. A., et al., 2006; Structural basis for HIV-1 neutralization by a gp41 fusion intermediate-directed antibody. Nat Struct Mol Biol 13:740-7), to highlight how the cholesterol group can bind to the membrane without perturbing antigen binding. Optimal antiviral activity is achieved by selecting a length of the linker between cysteine and cholesterol that is in the range of 50-150 Å.

### 2. Cholesterol Derivative of the HIV mAb 2F5.

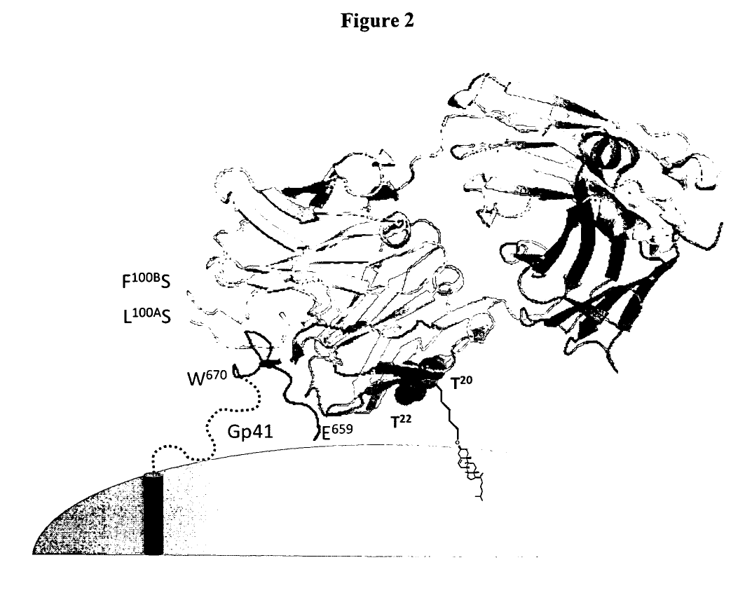

FIG. 2 shows the position of Thr²⁰ and Thr²² of the light chain in the crystal structure of the complex of mAb 2F5 with its peptide epitope (Thr²⁰ _{and Thr}²² represented as spheres). The complex is oriented relative to the viral membrane according to the currently accepted model (Ofek, G., et al., 2010; Relationship between Antibody 2F5 Neutralization of HIV-1 and Hydrophobicity of Its Heavy Chain Third Complementarity-Determining Region. J Virol 84:2955-2962; and according to Ofek, G., et al., 2004; Structure and mechanistic analysis of the anti-human immunodeficiency virus type 1 antibody 2F5 in complex with its gp41 epitope. J Virol 78:10724-37), to highlight how the cholesterol group can bind to the membrane without perturbing antigen binding. Optimal antiviral activity is achieved by selecting a length of the linker between cysteine and cholesterol that is in the range of 50-100 Å.

### 3. Cholesterol Derivative of the HIV mAb 4E10.

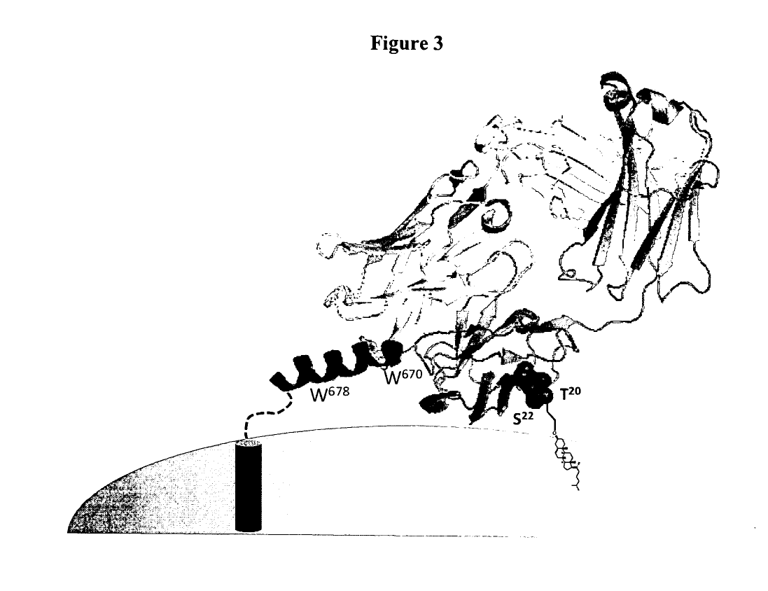

FIG. 3 shows the position of Thr²⁰ and Ser²² of the light chain in the crystal structure of the complex of mAb 4E10 with its peptide epitope (Thr²⁰ and Ser²² represented as spheres). The complex is oriented relative to the viral membrane according to the currently accepted model (Cardoso, R. M. F., et al., 2005; Broadly Neutralizing Anti-HIV Antibody 4E10 Recognizes a Helical Conformation of a Highly Conserved Fusion-Associated Motif in gp41 Immunity 22:163-173), to highlight how the cholesterol group can bind to the membrane without perturbing antigen binding. Optimal antiviral activity is achieved by selecting a length of the linker between cysteine and cholesterol that is in the range of 50-100 Å.

### 4. Cholesterol Derivative of the HIV mAb VRC01.

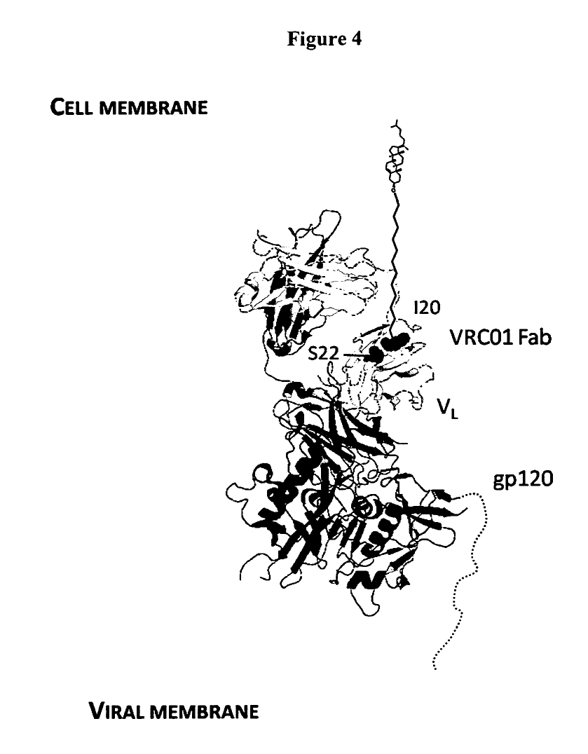

FIG. 4 shows the position of Ile²⁰ and Ser²² of the light chain in the crystal structure of the complex of mAb VRC01 with gp120 (Ile²⁰ and Ser²² represented as spheres). The complex is oriented relative to the viral membrane according to the currently accepted model (Zhu, T., et al., 2010; Structural Basis for Broad and Potent Neutralization of HIV-1 by Antibody VRC01. Science July 2010, DOI: 10.1126/science.1192819), to highlight how the cholesterol group can bind to the membrane without perturbing antigen binding. Optimal antiviral activity is achieved by selecting a length of the linker between cysteine and cholesterol that is in the range of 50-150 Å.

### 5. Cholesterol Derivative of the Influenza mAb CR6261.

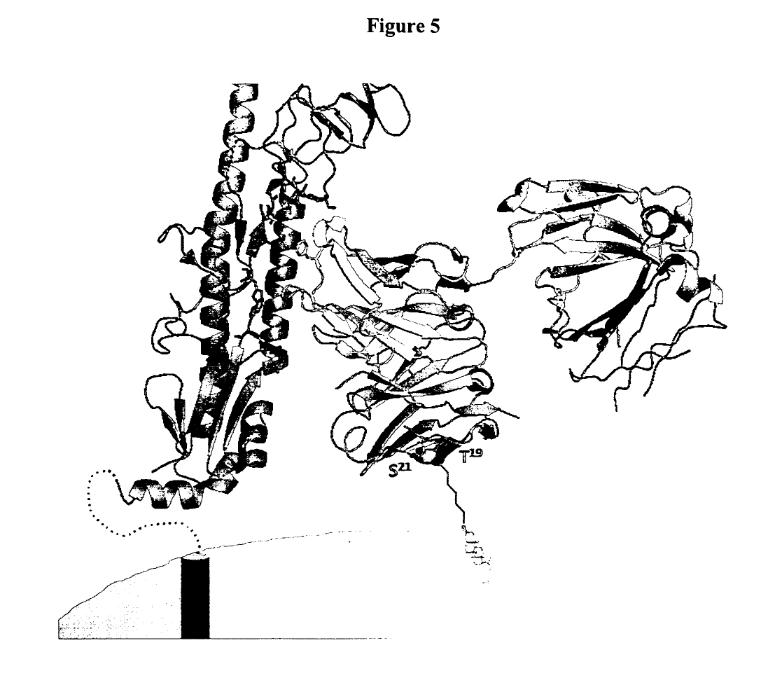

FIG. 5 shows the position of Thr¹⁹ and Ser²¹ of the light chain in the crystal structuᵣₑ of the complex of mAb CR6261 with the influenza hemagglutinin ³(Thr¹⁹ and Ser²¹ represented as spheres). The complex is oriented relative to the viral membrane according to the currently accepted model (Ekiert, D. C., et al., 2009; Antibody recognition of a highly conserved influenza virus epitope, Science, 324:246-51), to highlight how the cholesterol group can bind to the membrane without perturbing antigen binding. Optimal antiviral activity is achieved by selecting a length of the linker between cysteine and cholesterol that is in the range of 50-100 Å.

### 6. Cholesterol Derivative of Rituximab.

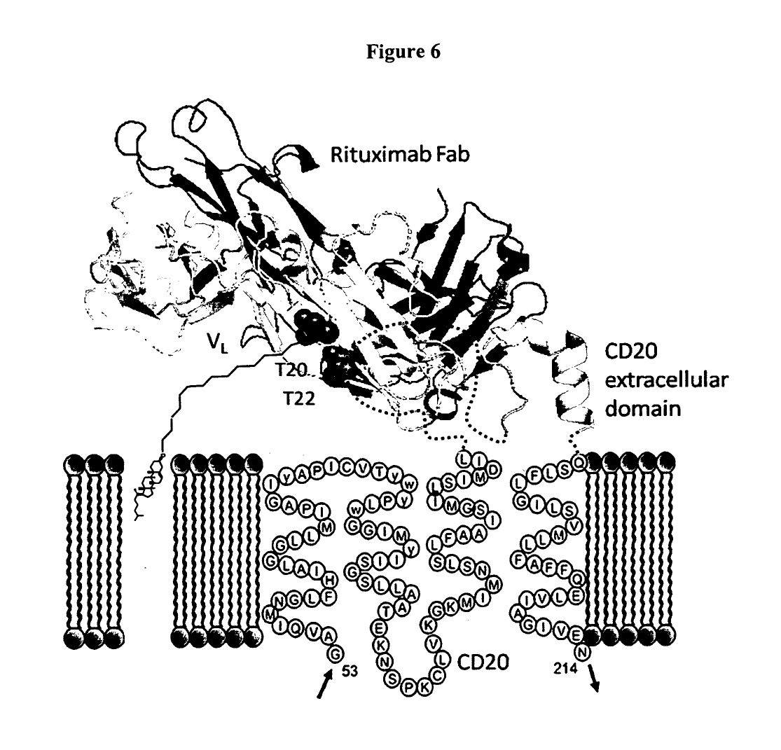

FIG. 6 shows the position of Thr²⁰ and Ser²² of the light chain in the crystal structure of the complex of the Rituximab Fab with a peptide corresponding to its epitope in the extracellular domain of CD20, as reported in Du, J. et al., 2007; Structural basis for recognition of CD20 by therapeutic antibody Rituximab, J. Biol. Chem., 282:15073-15080. The complex is oriented relative to the viral membrane according to the currently accepted model, to highlight how the cholesterol group can bind to the membrane without perturbing antigen binding. Optimal antiviral activity is achieved by selecting a length of the linker between cysteine and cholesterol that is in the range of 50-100 Å.

### 7. Cholesterol Derivative of Trastuzumab.

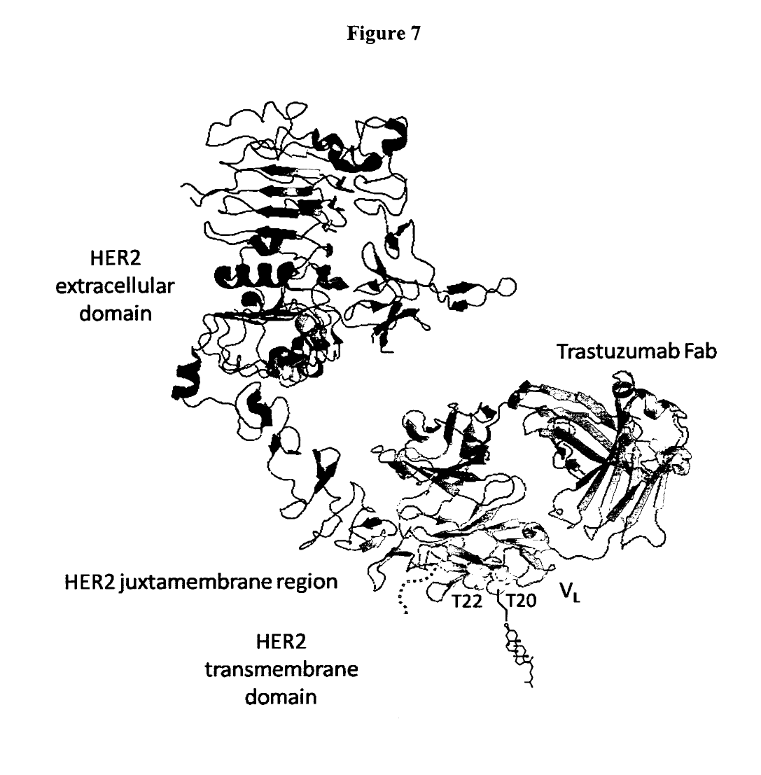

FIG. 7 shows the position of Thr²⁰ and Thr²² of the light chain in the crystal structure of the complex of the Trastuzumab Fab with the juxtamembrane region of the extracellular domain of HER2, as reported in Cho H.-S. et al., 2004; Structure of the extracellular region of HER2 alone and in complex with the Herceptin Fab, Nature, 421:756-60. Optimal antiviral activity is achieved by selecting a length of the linker between cysteine and cholesterol that is in the range of 50-100 Å.

### 8. Cholesterol Derivative of Cetuximab.

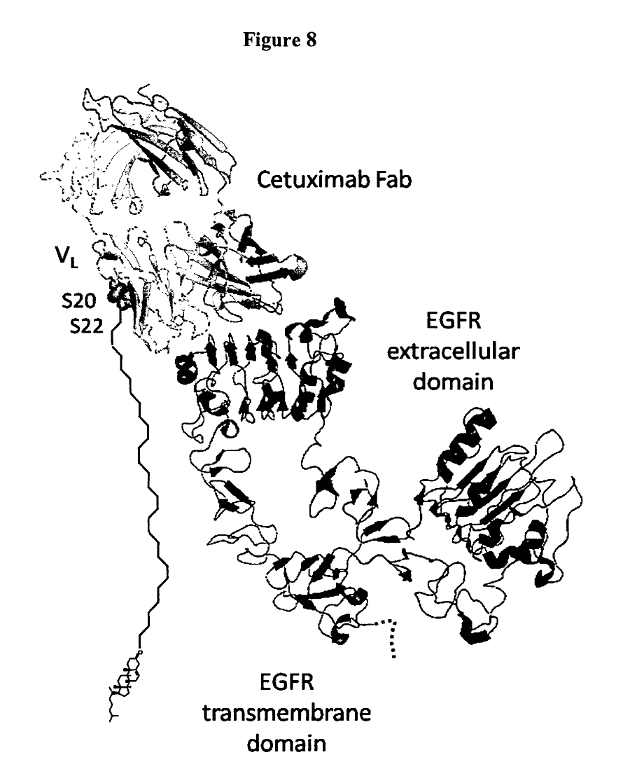

FIG. 8 shows the position of Thr²⁰ and Thr²² of the light chain in the crystal structure of the complex of the of the Cetuximab Fab with the extracellular domain of EGFR, as reported in Li, S. et al., 2005; Structural basis for inhibition of the Epidermal Growth Factor receptor by cetuximab, Cancer Cell, 7:301-311. Comparison with the structure of the Trastuzumab-HER2 complex (FIG. 7) illustrates the requirement for a different length of the linker. Optimal antiviral activity is achieved by selecting a length of the linker between cysteine and cholesterol that is in the range of 50-150 A.

### EXAMPLE 10

### Cholesterol Derivative of the HIV mAb D5

FIG. 1 shows the position of Thr²⁰ and Thr²² of the light chain in the crystal structure of the complex of mAb D5 with its peptide epitope (Thr²⁰ and Thr²² represented as spheres). The complex is oriented relative to the viral membrane according to the currently accepted model, to highlight how the cholesterol group can bind to the membrane without perturbing antigen binding. Optimal antiviral activity is achieved by selecting a length of the linker between cysteine and cholesterol that is in the range of 50-150 Å.

Unlike mAbs 2F5 and 4E10, which bind to the membrane-proximal external region (MPER) of gp41, close to the viral membrane, mAb D5 binds to a hydrophobic pocket in the HR1 domain of gp41, hence closer to the target cell membrane.

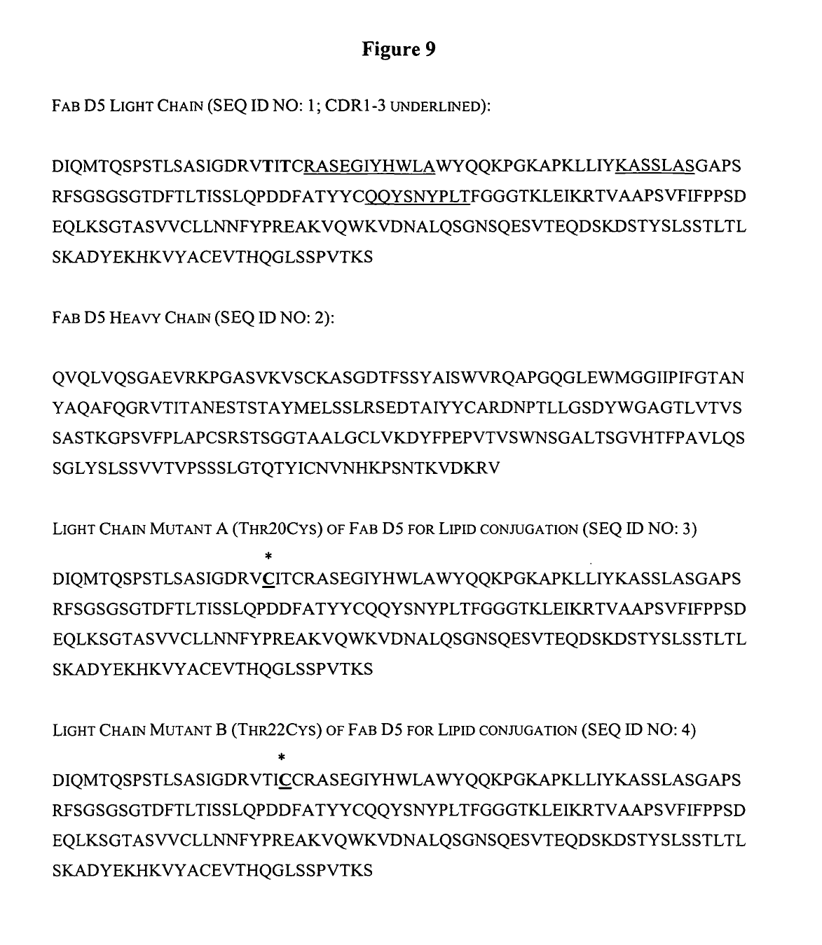

FIG. 9 illustrates the preferred and optimized locations of cysteine amino acids in the Fab domain of MAB D5, that are ideally positioned for covalent linkage to a lipid or a linker including a lipid according to the invention. (*) marks introduced cysteine amino acids.

### EXAMPLE 11

### Cholesterol Derivative of the HIV mAb 2F5

FIG. 2 shows the position of Thr²⁰ and Thr²² of the light chain in the crystal structure of the complex of mAb 2F5 with its peptide epitope (see: Ofek, G., et al., 2004; Structure and mechanistic analysis of the anti-human immunodeficiency virus type 1 antibody 2F5 in complex with its gp41 epitope, J Virol, 78:10724-37) (Thr²⁰ and Thr²² represented as spheres). The complex is oriented relative to the viral membrane according to the currently accepted model, to highlight how the cholesterol group can bind to the membrane without perturbing antigen binding. Optimal antiviral activity is achieved by selecting a length of the linker between cysteine and cholesterol that is in the range of 50-100 Å.

To test the concept that cholesterol would substitute the membrane raft binding activity of the natural 2F5 CDR3 loop, two mutations in the loop, which maintain intact the binding efficiency for the peptide epitope, but completely abolish the antiviral activity, are also introduced, as described in Ofek et al., J. Virol. 84 (2010) 2955-2962: Leu100_{A}Ser and Phe100_{B}Ser.

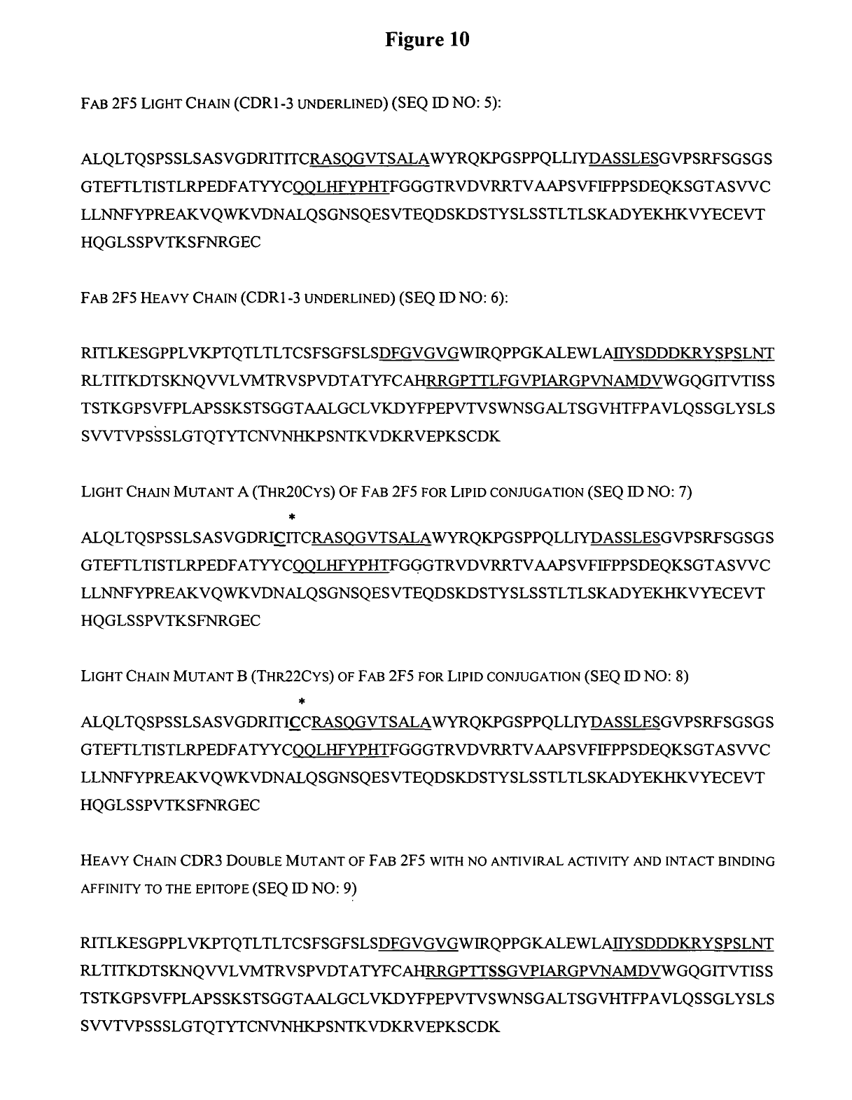

FIG. 10 illustrates the preferred and optimized locations of cysteine amino acids in the Fab domain of mAb 2F5, that are ideally positioned for covalent linkage to a lipid or a linker including a lipid according to the invention. Also shown is a double mutant of Fab 2F5 with no antiviral activity. (*) marks introduced cysteine amino acids.

### EXAMPLE 12

### Antiviral Activity of Antibodies

The antiviral activity of the antibodies was assessed for HIV as described in Miller et al., Proc. Natl. Acad. Sci. U.S.A. 102 (2005) 14759-14764, and Ingallinella et al., Proc. Natl. Acad. Sci. U.S.A. 106 (2009) 5801-5806, and for influenza as described in Throsby et al., PLoS ONE 3 (2008) e3942.

### EXAMPLE 13

### Conjugation of Cholesterol to the Anti-ErbB2 mAb Trastuzumab

Expression plasmids encoding for the heavy and light chains of the anti-ErbB2 mAb Trastuzumab (Herceptin®) were generated. An expression plasmid encoding for a mutated light chain of the anti-ErbB2 mAb Trastuzumab, featuring the substitution of Thr in position 20 with Cys (TrastuzumabC20, HerceptinC20) was also generated. Wild type and the T20→C TrastuzumabC20 mutant mAbs were produced by transient co-transfection of heavy and light expression plasmids into HEK-293 EBNA cells with Lipofectamine (Invitrogen), and the whole human IgGs were purified from culture medium with Hi-Trap protein A columns (Amersham Biosciences).

8 mg (1.15 ml) of TrastuzumabC20 mAb (7 mg/ml-45 μM) were incubated with 200-fold excess (9 mM) of L-cysteine (128 μl of 90 mM L-cysteine stock in TE pH 8.0) for 4 h at 37° C. in N2 atmosphere, followed by buffer exchange into TE pH 8.0. 6 mg of reduced TrastuzumabC20 (3.15 mg/ml-20 μM) were incubated with 10-fold molar excess of Cholesterol-PEPG₁₂-Maleimide (see structure below) dissolved in TE pH 8.0, 200 μM final) at room temperature for 45 minutes, followed by buffer exchange into PBS.

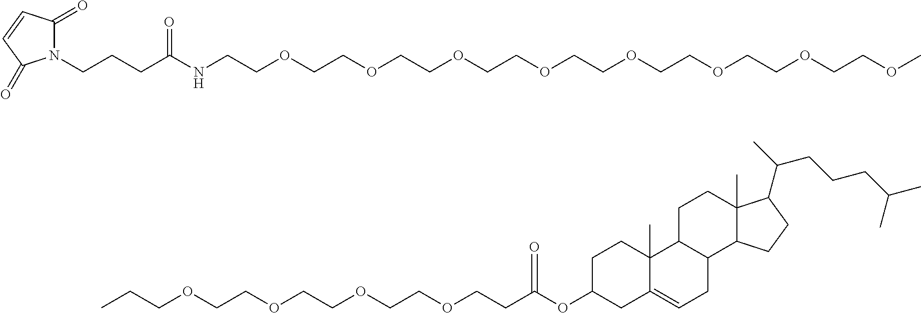

Conjugation of the cholesterol moiety to the mAb was confirmed by liquid chromatography-mass spectrometry (LC-MS) analysis. Prior to the analysis, the mAb was reduced in 6M guanidine hydrochloride, 0.1 M TRIS chloride buffer pH 8.4, with 5 mM DTT or 1% β-mercaptoethanol (90 min, 37° C.) and alkylated with 12.5 mM Iodoacetamide or 5 mM 4-vinylpyridine (90 min, room temp, in the dark). 2.5-5 μg of reduced and alkylated mAb were then injected onto a C8 RP column (ACE 2.1×50 mm, 300 Å, 5 μm) and eluted with a gradient of Acetonitrile in H₂O containing 5% methanol, 0.08% formic acid and 0.02% TFA. The samples were eluted directly into a Q-ToF MS hybrid system and analyzed.

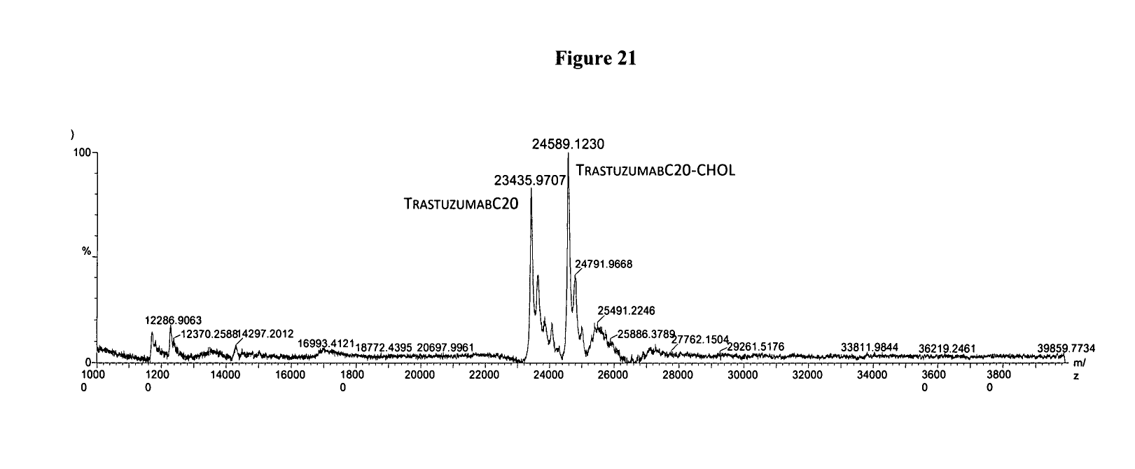

FIG. 21 shows a reconstructed MS spectrum of the light chain of the TrastuzumabC20 mAb after reaction with Cholesterol-PEPG₁₂-Maleimide. The two peaks at mass=23,436 and mass=24,589 correspond to the unconjugated and conjugated light chain after reduction and alkylation, respectively (calculated difference in mass: 1151.5, found: 1153).The results shown in FIG. 21 confirmed that more than 50% of the TrastuzumabC20 mAb was derivatized with cholesterol.

### EXAMPLE 14

### Binding of the Anti-ErbB2 mAb Trastuzumab Conjugated with Cholesterol to Target Antigen Displayed on Living Cells

ErbB2-positive SKBR3 cells (ATCC HTB-30) and ErbB2-negative A431 control cells (ATCC CRL-1555), harvested in nonenzymatic dissociation solution (Sigma), were washed and transferred to U-bottom microtitre plates (2×10⁵ cells per well). After blocking with PBS containing 3% bovine serum albumin (BSA), cells were incubated with 100 nM of purified antibody described in Example 13 in ELISA buffer (PBS/BSA 3%) and allowed to bind for 2 hours at room temperature. After centrifugation and removal of supernatants, the precipitated cells were washed twice in 200 μl of PBS, resuspended in 100 μl of ELISA buffer and incubated with peroxidase-conjugated anti-human IgG (Fc-specific) antibody (Sigma) for detection of TrastuzumabC20 (HerceptinC20) and TrastuzumabC20-CHOL (HerceptinC20-CHOL containing the PEG₁₂-Cholesterol moiety) antibodies of Example 13. After 1 h, the plates were centrifuged, washed with PBS, and reacted with 3,3′,5,5′-tetramethylbenzidine (TMB) (Sigma). Binding values were determined from the absorbance at 450 nm, and reported as the mean of at least three determinations (standard deviation <5%).

The results reported in FIG. 21 indicate an increase in the binding efficiency of the TrastuzumabC20-CHOL (HerceptinC20-CHOL) with respect to the unconjugated antibody. No binding was observed on ErbB2-negative A431 control cells.

1. An lipid-conjugated antibody or lipid-conjugated fragment thereof, wherein the antibody or the fragment thereof is covalently linked, optionally via a linker, to a lipid, wherein the lipid or said linker is covalently linked to an amino acid of an antibody domain of said antibody or fragment thereof selected from the group consisting of VL, VH, CL, CH1, CH2 and CH3 and wherein the antibody inhibits viral fusion.

2. The antibody or fragment thereof of claim 1, wherein the antibody or fragment thereof is capable of:
(i) being internalized into a cell;
(ii) binding to the lipid membrane of a cell and/or
(iii) binding to the lipid membrane of an enveloped virus.

3. The antibody or fragment thereof of claim 1 or 2, wherein the antibody binds to a polypeptide selected from the group consisting of HIV gp41, HIV gp120, influenza hemagglutinin, protein F of paramyxoviruses, protein GP2 of filoviruses, protein E of flaviviruses, protein S of coronaviruses and protein G2 of arenaviruses.

4. The antibody or fragment thereof of claim 1 or 2, wherein the antibody binds to a polypeptide associated to the plasma membrane and mediates binding and entry of a virus selected from the group consisting of retroviruses, influenza viruses, paramyxoviruses, filoviruses, flaviviruses, coronaviruses and arenaviruses.

5. (canceled)

6. The antibody or fragment thereof of claim 2, wherein the amino acid is located:
(1) N-terminal to the CDR-1 region of the VL domain of said antibody or fragment thereof;
(2) N-terminal to the CDR-1 region of the VH domain of said antibody or fragment thereof;
(3) within the CDR-3 region of the VL domain of said antibody or fragment thereof; or
(4) within the CDR-3 region of the VH domain of said antibody or fragment thereof.

7. The antibody or fragment thereof of claim 1 or 2, wherein the amino acid is located:
(1) at position 20 or 22 of the VL domain of said antibody or fragment thereof;
(2) at position 19 or 21 of the VL domain of said antibody or fragment thereof;
(3) at position 7 or 25 of the VH domain of said antibody or fragment thereof;
(4) at position 197 of the CL domain of said antibody or fragment thereof;
(5) at position 125 of the CH1 domain of said antibody or fragment thereof;
(6) at position 248 or 326 of the CH2 domain of said antibody or fragment thereof; or
(7) at position 415 or 442 of the CH3 domain of said antibody or fragment thereof.

8. The antibody or fragment thereof according to claim 1 or 2, wherein the lipid is selected from the group consisting of cholesterol, a sphingolipid, a glycolipid, a glycerophospholipid and a derivative or pharmaceutically acceptable salt thereof.

9. The antibody or fragment thereof according to claim 1 or 2, wherein said lipid is covalently linked to said antibody or fragment via a linker and wherein said linker, which is preferably a non-cleavable linker, has a length of between 0.4 nm and 15 nm.

10. The antibody or fragment thereof according to claim 1 or 2, wherein the linker or lipid is covalently linked to the antibody or fragment thereof via a bond selected from the group consisting of an amide bond, an ester bond, a thioether bond, a thioester bond, an aldehyde bond and an oxyme bond.

11. The antibody or fragment thereof according to claim 1 or 2, wherein the linker or lipid is covalently linked to a cysteine of said antibody or fragment thereof.

12. The antibody or fragment thereof according to claim 1 or 2, wherein the linker comprises or consists of a moiety selected from the group consisting of Y, —(CH₂)ₙ—, —(CH₂CH₂X)ₙ—, —(CH₂CH₂CH₂X)ₙ—, —Y—(CH₂CH₂X)ₙ—, —Y—(CH₂CH₂CH₂X)ₙ—, —Y—(CH₂CH₂X)ₙ—Z, —Y—(CH₂CH₂CH₂X)ₙ—Z, —Y—(CH₂CH₂X)ₙ—CH₂—Z, —Y‘(CH₂CH₂CH₂X)ₙ—CH₂—Z, —Y—(CH₂CH₂X)ₙ—CH₂—CH₂—Z, —Y—(CH₂CH₂CH₂X)ₙ—CH₂—CH₂—Z, a glycosylphosphatidylinositol (GPI), a polynucleotide, an amino acid, a polypeptide, a carbohydrate and combinations thereof;
wherein X is —O— or —NH—, Y is —NH—, —NH—(C═O)—, —(C═O)—NH—, —CH₂—(C═O)—NH— or —CH₂—, Z is —NH—(C═O)—, —(C═O)—, and n is an integer of 0 to 40.

13. The antibody or fragment thereof according to claim 1 or 2, wherein the lipid is cholesterol or a derivative thereof and wherein the lipid is attached directly or via the linker to the antibody or fragment thereof through the oxygen moiety at the 3 position of the cholesterol or derivative thereof.

14. The antibody or fragment thereof according to claim 1 or 2, wherein the lipid is cholesterol and the structure of the cholesterol and the linker moiety is as set out in formulas (V) to (XIV) 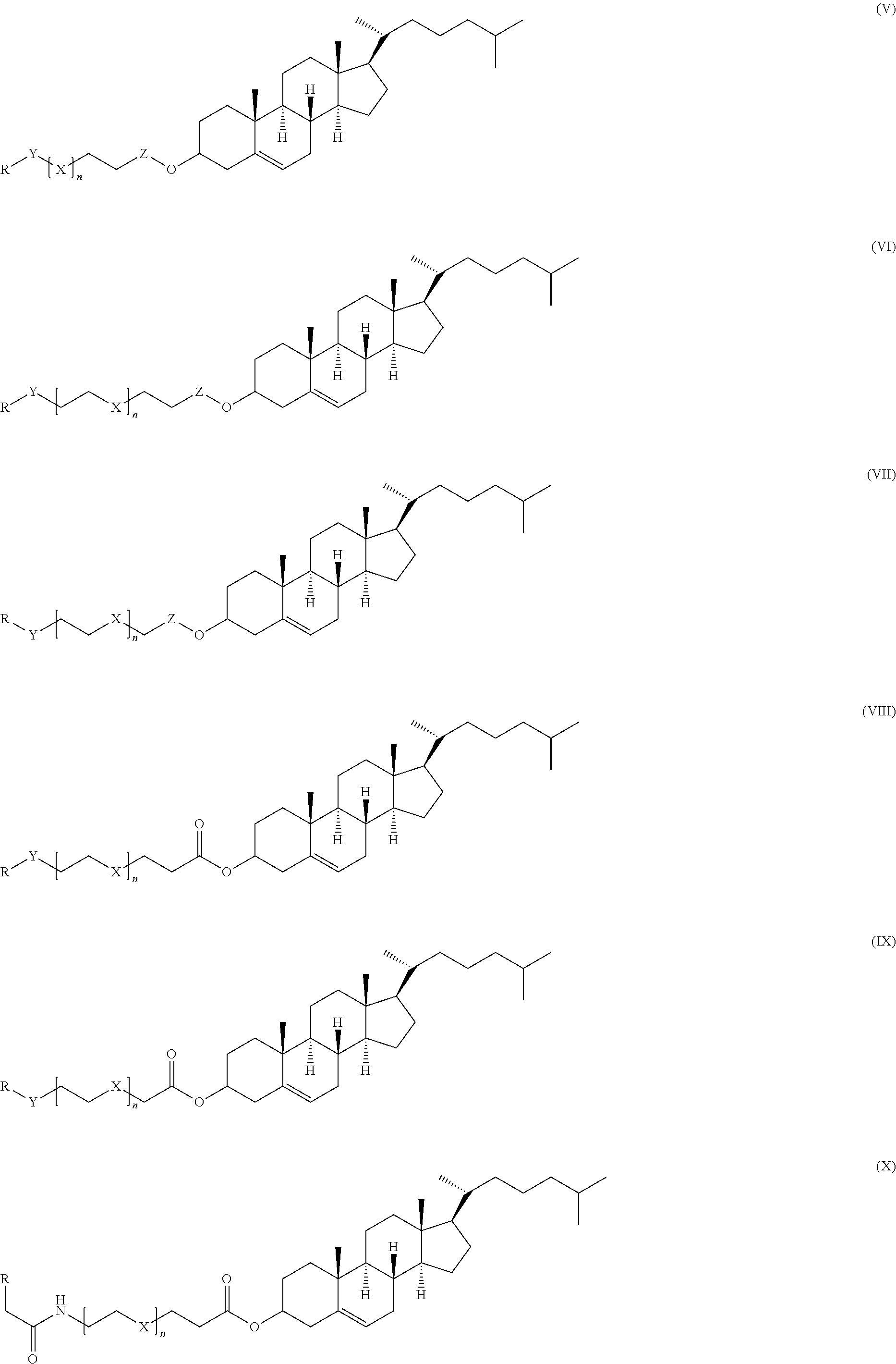 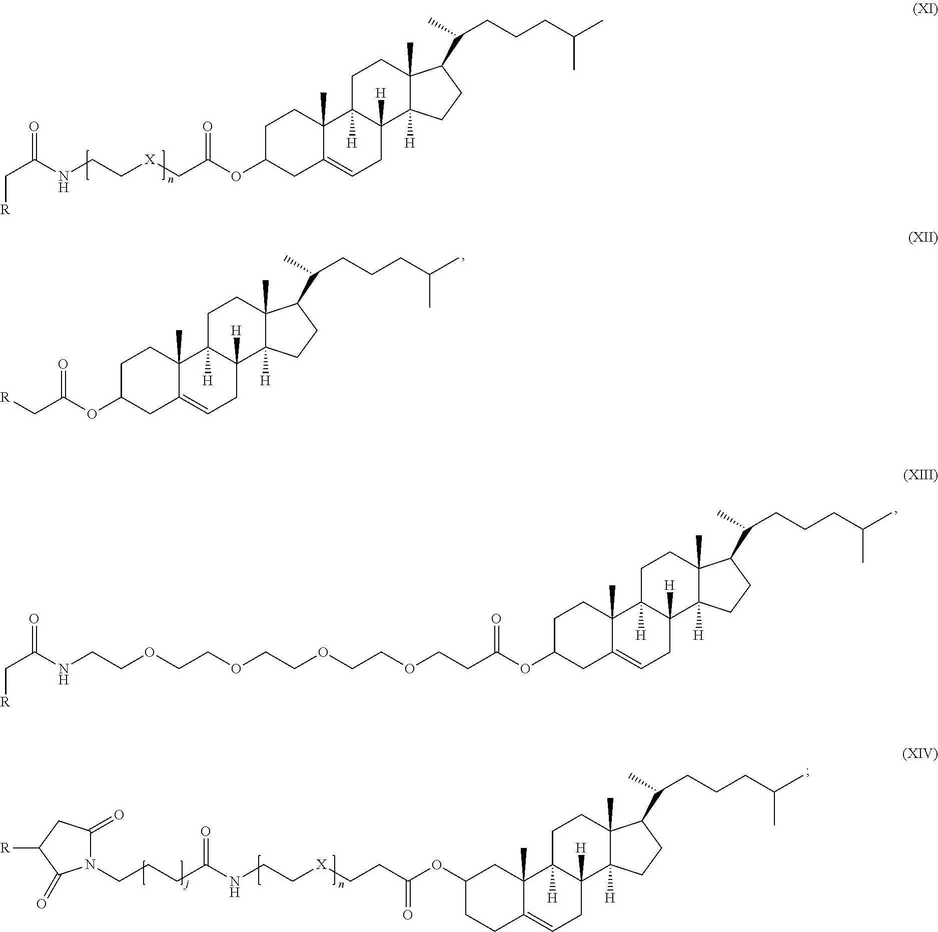
wherein X in each instance is independently selected from —NH—, —CH₂— and —O—; Y is selected from the group consisting of —CH₂—, —NH—, —NH—(C═O)—, —(C═O)—NH—, —CH₂—(C═O)—NH— and —CH₂—; Z is —NH—(C═O)—, —(C═O)—;
R designates the bond to the liker or to the antibody or fragment thereof, preferably a sulphur moiety of an amino acid thereof; and
n is an integer of 0 to 40; and
j is an integer selected from 0 to 40.

15. The antibody or fragment thereof according to claim 1 or 2, wherein the antibody is selected from a monoclonal antibody selected from the group consisting of MAB F10, MAB CR6261, MAB D5, MAB 2F5, MAB 4E10, MAB VRC01, MAB VRC0 2, palivizumab, motavizumab, wherein said monoclonal antibody optionally comprises one or two single amino acid substitutions, deletions, modifications and/or insertions.

16. The antibody or fragment thereof according to claim 1 or 2, wherein the CDR3 domain of the heavy chain of said antibody or fragment thereof comprises or consists of the sequence:

| | |
|---|---|
| RRGPTTXXXXXXARGPVNAMDV | (SEQ ID NO: 46) |
| or | |
| EGTTGXXXXXXPIGAFAH; | (SEQ ID NO: 47) |

wherein X may be any amino acid and wherein the lipid is covalently bound to one of the amino acids designated as X; and
wherein said sequence according to SEQ ID NO: 46 or 47 optionally comprises one single amino acid substitution, deletion, modification and/or insertion.

17. An antibody or fragment thereof of claim 1 or 2 for use in the treatment or the prevention of an infectious disease caused by viruses.

18. The antibody or fragment thereof of claim 17, wherein the disease caused by viruses is caused by a virus selected from the group consisting of HIV, Influenza virus, Hepatitis B virus, Hepatitis C virus, Rhinovirus, Herpes virus, Herpes simplex virus, West Nile Virus, Dengue virus, SARS-CoV, Varicella-zoster virus, Pseudorabies virus, Vesicular stomatits virus, Borna disease virus, Newcastle disease virus, Vaccinia virus, Rotavirus, Sendai virus, Measles virus, Mumps virus, Human Parainfluenza virus, Respiratory syncytical virus, Hendra virus, Nipah virus, Ebola virus, Marburg virus, Junin virus, Machupo virus, Guanairito virus, Lassa virus.
